(19) United States
(12) Patent Application Publication
(10) Pub. No.: US 2005/0288573 A1
(43) Pub. Date: Dec. 29, 2005

(54) Dectecting melanoma by electron paramagnetic resonance

(76) Inventor: Graham Stephen Timmins, Albuquerque, NM (US)

Correspondence Address: Schwegman, Lundberg, Woessner & Kluth, P.A., P.O. Box 2938, Minneapolis, MN, 55402, US

(21) Appl. No.: 11/142,631

(22) Filed: Jun. 1, 2005

Related U.S. Application Data
(60) Provisional application No. 60/576,403, filed on Jun. 1, 2004.
(60) Provisional application No. 60/609,599, filed on Sep. 14, 2004.
(60) Provisional application No. 60/613,860, filed on Sep. 28, 2004.

(51) Int. Cl. A61B 5/05

(52) U.S. Cl. 600/410

(57) ABSTRACT

Embodiments of methods and apparatus use electron paramagnetic resonance spectroscopy to provide a signal from melanin to image a melanoma. Embodiments of methods and apparatus use electron paramagnetic resonance spectroscopy to provide a signal from melanin to detect metastatic melanoma in a sentinel lymph node. Embodiments of methods and apparatus use electron paramagnetic resonance spectroscopy to provide a signal from melanin to measure light penetration in melanocytes in skin.

### RELATED APPLICATION

This application claims priority under 35 U.S.C. 119(e) from U.S. Provisional Application Ser. No. 60/576,403 filed 1 Jun. 2004, U.S. Provisional Application Ser. No. 60/609,599 filed 14 Sep. 2004, and U.S. Provisional Application Ser. No. 60/613,860 filed 28 Sep. 28, 2004, which applications are incorporated herein by reference.

### FIELD OF THE INVENTION

This invention relates generally to detecting melanoma using electron paramagnetic resonance.

### BACKGROUND OF THE INVENTION

Cutaneous malignant melanoma incidence continues to rise, with the American Cancer Society estimating 55,100 new cases of melanoma in 2004. Of these cases, about 7910 melanoma deaths will occur. The economic and emotional costs of this disease are large, with just the direct costs of treating newly diagnosed melanoma cases in 1997 alone being estimated at $546 Million. Furthermore, it is still unclear even exactly how sunlight exposure is linked to melanoma and these uncertainties have made it difficult to offer precise melanoma prevention recommendations. It seems certain, therefore, that the prevalence of melanoma, and the many costs associated with this disease, is high for many years to come.

One of the keys to improved melanoma outcomes is improved detection and diagnosis, so that suspect lesions are clinically evaluated better and earlier. However, despite continual advances, the overall diagnostic accuracy for melanoma has remained much lower that desirable for a disease with such high potential mortality rates, varying from 64% to 80%. While the index of suspicion for pigmented skin lesions remains high, lowering the numbers of false negatives (i.e. untreated melanomas) these will still occur, and the high numbers of false positives are associated with significant costs including unnecessary procedures, emotional distress and scarring etc. The sub-optimal accuracy of conventional diagnostic techniques has prompted the successful development of various imaging modalities for use in detecting and diagnosing melanoma, an area that has recently been reviewed very comprehensively.

These imaging techniques for detecting, diagnosing and staging melanoma are predominantly based upon optical techniques, as the melanin pigment in melanoma provides an inherent high contrast. However, a major limitation of optical techniques is that they cannot image deeply into the skin, as a consequence of both light scattering and absorbance, especially in highly pigmented lesions, and are limited to maximal depths of 0.2 to 0.3 mm. This is a major disadvantage, as tumor thickness is the single most powerful staging factor, and predictor of survival, with the important range of tumor thickness allowing this diagnostic prediction being from 0.5 to 10 mm. Therefore, this most important parameter is currently only available after excision. Although other imaging modalities such as computed tomography (CT) scanning, ultrasound, and magnetic resonance imaging (MRI) can image deeper melanomas, they are severely limited by lack of soft tissue contrast and/or melanoma specificity, in addition to a range of technique-specific technical difficulties. In addition to its optical properties, melanin contains a high concentration of stable free radicals formed during oxidation of its aromatic amino acid side-chains, so that Electron Paramagnetic Resonance (EPR) spectroscopy of these radicals is a useful technique for characterizing melanin.

### LITERATURE

The following publications provide additional information:
1. Grin, C., Kopf, A., Welkovich, B., Bar, R. & Levenstein, M. Accuracy in the Clinical-Diagnosis of Malignant-Melanoma. ARCHIVES OF DERMATOLOGY 126, 763-766 (1990).
2. Nachbar, F. et al. The Abcd Rule of Dermatoscopy—High Prospective Value in the Diagnosis of Doubtful Melanocytic Skin-Lesions. JOURNAL OF THE AMERICAN ACADEMY OF DERMATOLOGY 30, 551-559 (1994).
3. Marghoob, A. et al. Instruments and New Technologies for the in Vivo Diagnosis of Melanoma. JOURNAL OF THE AMERICAN ACADEMY OF DERMATOLOGY 49, 777-797 (2003).
4. Balch, C. M. et al. Prognostic Factors Analysis of 17,600 Melanoma Patients: Validation of the American Joint Committee on Cancer Melanoma Staging System. JOURNAL OF CLINICAL ONCOLOGY 19, 3622-3634 (2001).
5. Enochs, W., Nilges, M. & Swartz, H. A Standardized Test for the Identification and Characterization of Melanins Using Electron-Paramagnetic-Resonance (Epr) Spectroscopy. PIGMENT CELL RESEARCH 6, 91-99 (1993).
6. Sarna, T. & Swartz, H. Identification and Characterization of Melanin in Tissues and Body-Fluids. Folia Histochemica Et Cytochemica 16, 275-286 (1978).
7. Longstreth, J. Cutaneous Malignant Melanoma and Ultraviolet Radiation: A Review. Cancer & Metastasis Reviews 7, 321-33 (1988).
8. Woodhead, A. D., Setlow, R. B. & Tanaka, M. Environmental Factors in Nonmelanoma and Melanoma Skin Cancer. Journal of Epidemiology 9, S102-14 (1999).
9. Tsao, H., Rogers, G. & Sober, A. An Estimate of the Annual Direct Cost of Treating Cutaneous Melanoma. JOURNAL OF THE AMERICAN ACADEMY OF DERMATOLOGY 38, 669-680 (1998).
10. Wang, S. et al. Ultraviolet a and Melanoma: A Review. JOURNAL OF THE AMERICAN ACADEMY OF DERMATOLOGY 44,837-846 (2001).
11. Garland, C., Garland, F. & Gorham, E. Epidemiologic Evidence for Different Roles of Ultraviolet a and B Radiation in Melanoma Mortality Rates. Annals Of Epidemiology 13, 395-404 (2003).
12. Armstrong, B. K. & Kricker, A. The Epidemiology of Uv Induced Skin Cancer. Journal of Photochemistry and Photobiology B-Biology 63, 8-18 (2001).
13. Armstrong, B. K. & Kricker, A. How Much Melanoma Is Caused by Sun Exposure? MELANOMA RESEARCH 3, 395-401 (1993).
14. Armstrong, B. K. Epidemiology of Malignant Melanoma: Intermittent or Total Accumulated Exposure to the Sun? Journal of Dermatologic Surgery & Oncology 14, 835-49 (1988).
15. Margolis, D. J. et al. Validation of a Melanoma Prognostic Model. ARCHIVES OF DERMATOLOGY 134, 1597-601 (1998).
16. Marghoob, A., Koenig, K., Bittencourt, F., Kopf, A. & Bart, R. Breslow Thickness and Clark Level in Melanoma—Support for Including Level in Pathology Reports and in American Joint Committee on Cancer Staging. CANCER 88, 589-595 (2000).
17. Balch, C. M. et al. Final Version of the American Joint Committee on Cancer Staging System for Cutaneous Melanoma. JOURNAL OF CLINICAL ONCOLOGY 19, 3635-48 (2001).
18. Zitelli, J., Brown, C. & Hanusa, B. Surgical Margins for Excision of Primary Cutaneous Melanoma. JOURNAL OF THE AMERICAN ACADEMY OF DERMATOLOGY 37, 422-429 (1997).
19. Balch, C. M. et al. Long-Term Results of a Prospective Surgical Trial Comparing 2 Cm Vs. 4 Cm Excision Margins for 740 Patients with 1-4 Mm Melanomas. [See Comment]. Annals of Surgical Oncology 8, 101-8 (2001).
20. Aitken, J. et al. Reliability of Computer Image Analysis of Pigmented Skin Lesions of Australian Adolescents. CANCER 78, 252-257 (1996).
21. Yang, P. et al. Macroscopic Spectral Imaging and Gene Expression Analysis of the Early Stages of Melanoma. MOLECULAR MEDICINE 5, 785-794 (1999).
22. Farkas, D. & Becker, D. Applications of Spectral Imaging: Detection and Analysis of Human Melanoma and Its Precursors. PIGMENT CELL RESEARCH 14, 2-8 (2001).
23. Busam, K. et al. Detection of Intraepidermal Malignant Melanoma in Vivo by Confocal Scanning Laser Microscopy. MELANOMA RESEARCH 12, 349-355 (2002).
24. Welzel, J. Optical Coherence Tomography in Dermatology: A Review. SKIN RESEARCH AND TECHNOLOGY 7, 1-9 (2001).
25. Mcgregor, B. Automatic Registration of Images of Pigmented Skin Lesions. PATTERN RECOGNITION 31, 805-817 (1998).
26. Jemec, G. B., Gniadecka, M. & Ulrich, J. Ultrasound in Dermatology. Part I. High Frequency Ultrasound. European Journal of Dermatology 10, 492-7 (2000).
27. Veronesi, U. et al. Inefficacy of Immediate Node Dissection in Stage 1 Melanoma of the Limbs. New England Journal of Medicine 297, 627-30 (1977).
28. Veronesi, U. et al. Delayed Regional Lymph Node Dissection in Stage I Melanoma of the Skin of the Lower Extremities. CANCER 49, 2420-30 (1982).
29. Sim, F. H., Taylor, W. F., Pritchard, D. J. & Soule, E. H. Lymphadenectomy in the Management of Stage I Malignant Melanoma: A Prospective Randomized Study. Mayo Clinic Proceedings 61, 697-705 (1986).
30. Sim, F. H., Taylor, W. F., Ivins, J. C., Prtchard, D. J. & Soule, E. H. A Prospective Randomized Study of the Efficacy of Routine Elective Lymphadenectomy in Management of Malignant Melanoma. Preliminary Results. CANCER 41, 948-56 (1978).
31. Balch, C. M. et al. Long-Term Results of a Multi-Institutional Randomized Trial Comparing Prognostic Factors and Surgical Results for Intermediate Thickness Melanomas (1.0 to 4.0 Mm). Intergroup Melanoma Surgical Trial. [See Comment]. Annals of Surgical Oncology 7, 87-97 (2000).
32. Balch, C. M. et al. Efficacy of an Elective Regional Lymph Node Dissection of 1 to 4 Mm Thick Melanomas for Patients 60 Years of Age and Younger. Annals of Surgery 224, 255-63; discussion 263-6 (1996).
33. Dessureault, S. et al. Improved Staging of Node-Negative Patients with Intermediate to Thick Melanomas (>1 Mm) with the Use of Lymphatic Mapping and Sentinel Lymph Node Biopsy. [See Comment] [Erratum Appears in Ann Surg Oncol 2002 April; 9(3):318]. Annals of Surgical Oncology 8, 766-70 (2001).
34. Carlson, G. W. et al. Sentinel Lymph Node Mapping for Thick (> or =4-Mm) Melanoma: Should We Be Doing It? Annals of Surgical Oncology 10, 408-15 (2003).
35. Mcmasters, K. M. et al. Interval Sentinel Lymph Nodes in Melanoma. Archives of Surgery 137, 543-7; discussion 547-9 (2002).
36. Mcmasters, K. M. et al. Sentinel Lymph Node Biopsy for Melanoma: Controversy Despite Widespread Agreement. JOURNAL OF CLINICAL ONCOLOGY 19, 2851-5 (2001).
37. Kalady, M. F. et al. Validation of Delayed Sentinel Lymph Node Mapping for Melanoma. Cancer Journal 7, 503-8 (2001).
38. Bachter, D., Michl, C., Buchels, H., Vogt, H. & Balda, B. R. The Predictive Value of the Sentinel Lymph Node in Malignant Melanomas. Recent Results in Cancer Research 158, 129-36 (2001).
39. Medalie, N. S. & Ackerman, A. B. Sentinel Lymph Node Biopsy Has No Benefit for Patients with Primary Cutaneous Melanoma Metastatic to a Lymph Node: An Assertion Based on Comprehensive, Critical Analysis: Part I. AMERICAN JOURNAL OF DERMATOPATHOLOGY 25,399-417 (2003).
40. Agnese, D. M. et al. Cost-Effectiveness of Sentinel Lymph Node Biopsy in Thin Melanomas. Surgery 134, 542-7; discussion 547-8 (2003).
41. Stojadinovic, A., Allen, P. J., Clary, B. M., Busam, K. J. & Coit, D. G. Value of Frozen-Section Analysis of Sentinel Lymph Nodes for Primary Cutaneous Malignant Melanoma. Annals of Surgery 235, 92-8 (2002).
42. Longo, M. I., Lazaro, P., Bueno, C., Carreras, J. L. & Montz, R. Fluorodeoxyglucose-Positron Emission Tomography Imaging Versus Sentinel Node Biopsy in the Primary Staging of Melanoma Patients. Dermatologic Surgery 29, 245-8 (2003).
43. Belhocine, T., Pierard, G., De Labrassinne, M., Lahaye, T. & Rigo, P. Staging of Regional Nodes in Ajcc Stage I and Ii Melanoma: 18fdg Pet Imaging Versus Sentinel Node Detection. Oncologist 7, 271-8 (2002).
44. Davids, A., Kidsona, E. & Hanekomb, G. Melanoma Patient Staging: Histopathological Versus Molecular Evaluation of the Sentinel Node. MELANOMA RESEARCH 13, 313-324 (2003).
45. Ito, S. & Wakamatsu, K. Quantitative Analysis of Eumelanin and Pheomelanin in Humans, Mice and Other Animals: A Comparative Review. PIGMENT CELL RESEARCH 16, 523-531 (2003).
46. Morishima, T. & Fukuda, E. Quantitative Analysis of Eumelanin and Pheomelanin in Human Malignant-Melanoma Tissues. Archives of Dermatological Research 277, 248-250 (1985).
47. Collins, B., Poehler, T. O. & Bryden, W. A. Epr Persistence Measurements of Uv-Induced Melanin Free Radicals in Whole Skin. Photochemistry & Photobiology 62, 557-60 (1995).
48. Atlas, S. W., Braffman, B. H., Lobrutto, R., Elder, D. E. & Herlyn, D. Human Malignant Melanomas with Varying Degrees of Melanin Content in Nude Mice: Mr Imaging, Histopathology, and Electron Paramagnetic Resonance. Journal of Computer Assisted Tomography 14, 547-54 (1990).
49. Elek, G., Lapis, K. & Rockenbauer, A. Esr Investigation of Paraffin-Embedded Ocular Melanomas. BRITISH JOURNAL OF CANCER 41, 199-203 (1980).
50. Katsuda, H., Kobayashi, T., Saito, H., Matsunaga, T. & Ikeya, M. Electron Spin Resonance Imaging of Mouse B16 Melanoma. Chemical & Pharmaceutical Bulletin 38, 2838-40 (1990).
51. Eaton, G., Eaton, S. & Ohno, K. Epr Imaging and in Vivo Epr (CRC press, Boca Raton, 1991).
52. Kuppusamy, P. et al. 3-Dimensional Spectral Spatial Epr Imaging of Free-Radicals in the Heart—a Technique for Imaging Tissue Metabolism and Oxygenation. PROCEEDINGS OF THE NATIONAL ACADEMY OF SCIENCES OF THE UNITED STATES OF AMERICA 91, 3388-3392 (1994).
53. Alecci, M., Ferrari, M., Quaresima, V., Sotgiu, A. & Ursini, C. Simultaneous 280-Mhz Epr Imaging of Rat Organs During Nitroxide Free-Radical Clearance. BIOPHYSICAL JOURNAL 67, 1274-1279 (1994).
54. Halpern, H. et al. In Vivo Spin-Label Murine Pharmacodynamics Using Low-Frequency Electron Paramagnetic Resonance Imaging. BIOPHYSICAL JOURNAL 71, 403-409 (1996).
55. Murugesan, R. et al. In Vivo Imaging of a Stable Paramagnetic Probe by Pulsed-Radiofrequency Electron Paramagnetic Resonance Spectroscopy. MAGNETIC RESONANCE IN MEDICINE 38, 409-414 (1997).
56. Togashi, H. et al. Spatiotemporal Measurement of Free Radical Elimination in the Abdomen Using an in Vivo Esr-Ct Imaging System. FREE RADICAL BIOLOGY AND MEDICINE 25, 1-8 (1998).
57. Eaton, S. & Eaton, G. in Electron Paramagnetic Resonance 17 (eds. Gilbert, B., Davies, M. & McLauchlan, K.) 109-129 (Royal Society of Chemistry, Cambridge, 2000).
58. Hutchison, J. M. S. in Biological Magnetic Resonance 18; in Vivo Epr (Esr), Theory and Applications (ed. Berliner, L. J.) 41-48 (Kluwer, New York, 2003).
59. Lucarini, M., Pedulli, G., Motyakin, M. & Schlick, S. Electron Spin Resonance Imaging of Polymer Degradation and Stabilization. Progress in Polymer Science 28, 331-340 (2003).
60. Bacic, G., Nilges, M. J., Magin, R. L., Walczak, T. & Swartz, H. M. In Vivo Localized Esr Spectroscopy Reflecting Metabolism. Magnetic Resonance in Medicine 10, 266-72 (1989).
61. Swartz, H. M. et al. Measurements of Pertinent Concentrations of Oxygen in Vivo. Magnetic Resonance in Medicine 20, 333-9 (1991).
62. Iannone, A. et al. Blood Clearance of Dextran Magnetite Particles Determined by a Noninvasive in Vivo Esr Method. Magnetic Resonance in Medicine 22, 435-42 (1991).
63. Liu, K. J. et al. Lithium Phthalocyanine: A Probe for Electron Paramagnetic Resonance Oximetryin Viable Biological Systems. Proceedings of the National Academy of Sciences of the United States of America 90, 5438-42 (1993).
64. Swartz, H. M., Liu, K. J., Goda, F. & Walczak, T. India Ink: A Potential Clinically Applicable Epr Oximetry Probe. Magnetic Resonance in Medicine 31, 229-32 (1994).
65. Smirnov, A. I., Norby, S. W., Walczak, T., Liu, K. J. & Swartz, H. M. Physical and Instrumental Considerations in the Use of Lithium Phthalocyanine for Measurements of the Concentration of the Oxygen. Journal of Magnetic Resonance Series B 103, 95-102 (1994).
66. Noonan, F. P. et al. Neonatal Sunburn and Melanoma in Mice. NATURE 413, 271-2 (2001).
67. Takayama, H. et al. Diverse Tumorigenesis Associated with Aberrant Development in Mice Overexpressing Hepatocyte Growth Factor Scatter Factor. Proceedings of the National Academy of Sciences of the United States of America 94, 701-706 (1997).
68. Noonan, F., Dudek, J., Merlino, G. & De Fabo, E. Animal Models of Melanoma: An Hgf/Sf Transgenic Mouse Model May Facilitate Experimental Access to Uv Initiating Events. PIGMENT CELL RESEARCH 16, 16-25 (2003).
69. Kusewitt, D. F., Applegate, L. A., Bucana, C. D. & Ley, R. D. Naturally Occurring Malignant Melanoma in the South American Opossum (Monodelphis Domestica). Veterinary Pathology 27, 66-8 (1990).
70. Kusewitt, D. F., Applegate, L. A. & Ley, R. D. Ultraviolet Radiation-Induced Skin Tumors in a South American Opossum (Monodelphis Domestica). Veterinary Pathology 28, 55-65 (1991).
71. Kusewitt, D. F., Miska, K. B. & Ley, R. D. S-100 Immunoreactivity in Melanomas of the South American Opossum Monodelphis Domestica. Veterinary Pathology 34, 346-50 (1997).
72. Ley, R. D. Photoreactivation of Uv-Induced Pyrimidine Dimers and Erythema in the Marsupial Monodelphis Domestica. Proceedings of the National Academy of Sciences of the United States of America 82, 2409-11 (1985).
73. Ley, R. D., Applegate, L. A., Stuart, T. D. & Fry, R. J. Uv Radiation-Induced Skin Tumors in Monodelphis Domestica. Photo-Dermatology 4, 144-7 (1987).
74. Ley, R. D., Applegate, L. A., Padilla, R. S. & Stuart, T. D. Ultraviolet Radiation—Induced Malignant Melanoma in Monodelphis Domestica. Photochemistry & Photobiology 50, 1-5 (1989).
75. Ley, R. D. Ultraviolet Radiation a-Induced Precursors of Cutaneous Melanoma in Monodelphis Domestica. Cancer Research 57, 3682-4 (1997).
76. Ley, R. D., Reeve, V. E. & Kusewitt, D. F. Photobiology of Monodelphis Domestica. Developmental & Comparative Immunology 24, 503-16 (2000).
77. Ley, R. D. Dose Response for Ultraviolet Radiation a-Induced Focal Melanocytic Hyperplasia and Nonmelanoma Skin Tumors in Monodelphis Domestica. Photochemistry & Photobiology 73, 20-3 (2001).
78. Ley, R. D. Animal Models of Ultraviolet Radiation (Uvr)-Induced Cutaneous Melanoma. Frontiers in Bioscience 7, d1531-4 (2002).
79. Pathak, S., Gadhia, M., Dhaliwal, M. K., Applegate, L. A. & Ley, R. D. Specific Chromosomal Defects Associated with Ultraviolet Radiation-Induced Cutaneous Tumors in Monodelphis Domestica (Marsupialia, Mammalia). Cancer Genetics & Cytogenetics 64, 158-62 (1992).
80. Robinson, E., Vandeberg, J., Hubbard, G. & Dooley, T. Malignant-Melanoma in Ultraviolet-Irradiated Laboratory Opossums—Initiation in Suckling Young, Metastasis in Adults, and Xenograft Behavior in Nude-Mice. CANCER RESEARCH 54, 5986-5991 (1994).
81. Robinson, E., Hubbard, G. & Dooley, T. Metastatic Melanoma in an Adult Opossum (Monodelphis Domestica) after Short-Term Intermittent Uvb Exposure. Archives of Dermatological Research 292, 469-471 (2000).
82. Vandeberg, J. L., Williams-Blangero, S., Hubbard, G. B., Ley, R. D. & Robinson, E. S. Genetic Analysis of Ultraviolet Radiation-Induced Skin Hyperplasia and Neoplasia in a Laboratory Marsupial Model (Monodelphis Domestica). Archives of Dermatological Research 286, 12-7 (1994).
83. Wang, Z., Hubbard, G., Pathak, S. & Vandeberg, J. In Vivo Opossum Xenograft Model for Cancer Research. CANCER RESEARCH 63, 6121-6124 (2003).
84. Robinson, E. S., Hil, R. H., Jr., Kripke, M. L. & Setlow, R. B. The Monodelphis Melanoma Model: Initial Report on Large Ultraviolet a Exposures of Suckling Young. Photochemistry & Photobiology 71, 743-6 (2000).
85. Liu, Y. & Simon, J. D. Isolation and Biophysical Studies of Natural Eumelanins: Applications of Imaging Technologies and Ultrafast Spectroscopy. PIGMENT CELL RESEARCH 16, 606-618 (2003).
86. Setlow, R. B., Grist, E., Thompson, K. & Woodhead, A. D. Wavelengths Effective in Induction of Malignant Melanoma. Proceedings of the National Academy of Sciences of the United States of America 90(14), 6666-70 (1993).
87. Ahmed, F. E. & Setlow, R. B. Ultraviolet Radiation-Induced DNA Damage and Its Photorepair in the Skin of the Platyfish Xiphophorus. CANCER RESEARCH 53, 2249-55 (1993).
88. Setlow, R. B., Woodhead, A. D. & Grist, E. Animal Model for Ultraviolet Radiation-Induced Melanoma: Platyfish-Swordtail Hybrid. Proceedings of the National Academy of Sciences of the United States of America 86, 8922-6 (1989).
89. Jackson, S. K., Madhani, M., Thomas, M., Timmins, G. S. & James, P. E. Applications of in Vivo Electron Paramagnetic Resonance (Epr) Spectroscopy: Measurements of Po2 and No in Endotoxin Shock. Toxicology Letters 120, 253-7 (2001).
90. Timmins, G., Penatti, C., Bechara, E. & Swartz, H. Measurement of Oxygen Partial Pressure, Its Control During Hypoxia and Hyperoxia, and Its Effect Upon Light Emission in a Bioluminescent Elaterid Larva. JOURNAL OF EXPERIMENTAL BIOLOGY 202, 2631-2638 (1999).
91. Collier, J. P. et al. Comparison of Cross-Linked Polyethylene Materials for Orhopaedic Applications. Clinical Orthopaedics and Related Research 414, 289-304 (2003).
92. Timmins, G. S., Liu, K. J., Bechara, E. J., Kotake, Y. & Swartz, H. M. Trapping of Free Radicals with Direct in Vivo Epr Detection: A Comparison of 5,5-Dimethyl-1-Pyrroline-N-Oxide and 5-Diethoxyphosphoryl-5-Methyl-1-Pyrroline-N-Oxide as Spin Traps for Ho* and S04*. Free Radical Biology & Medicine 27, 329-33 (1999).
93. Timmins, G. S. & Davies, M. J. Free Radical Formation in Murine Skin Treated with Tumour Promoting Organic Peroxides. Carcinogenesis 14, 1499-503 (1993).
94. Timmins, G. S. & Davies, M. J. Free Radical Formation in Isolated Murine Keratinocytes Treated with Organic Peroxides and Its Modulation by Antioxidants. Carcinogenesis 14, 1615-20 (1993).
95. He, G. et al. Mapping of the B1 Field Distribution of a Surface Coil Resonator Using Epr Imaging. Magnetic Resonance in Medicine 48, 1057-1062 (2002).
96. Berliner, L. & Fujii, H. Magnetic-Resonance Imaging of Biological Specimens by Electron-Paramagnetic Resonance of Nitroxide Spin Labels. SCIENCE 227, 517-519 (1985).
97. Queirolo, P., Taveggia, P., Gipponi, M. & Sertoli, M. R. Sentinel Lymph Node Biopsy in Melanoma Patients: The Medical Oncologist's Perspective. Journal of Surgical Oncology 85, 162-5 (2004).
98. Korytowski, W., et al., Photoinduced Generation of Hydrogen Peroxide and Hydroxyl Radicals in Melanin. Photochemistry and Photobiology, 1987. 45(2): p. 185-190.
99. Sarna, T., I. A. Menon, and R. C. Sealy. Photoinduced Oxygen-Consumption in Melanin Systems .2. Action Spectra and Quantum Yields for Pheomelanins. Photochemistry and Photobiology, 1984. 39(6): p. 805-809.
100. Sarna, T. and R. C. Sealy, Free-Radicals from Eumelanins: Quantum Yields and Wavelength Dependence. Archives of Biochemistry and Biophysics, 1984. 232(2): p. 574-578.
101. Sarna, T. and R. C. Sealy, Photoinduced Oxygen-Consumption in Melanin Systems. Action Spectra and Quantum Yields for Eumelanin and Synthetic Melanin. Photochemistry and Photobiology, 1984. 39(1): p. 69-74.
102. Kipp, C. and A. R. Young, The Soluble Eumelanin Precursor 5,6-Dihydroxyindole-2-Carboxylic Acid Enhances Oxidative Damage in Human Keratinocyte DNA after Uva Irradiation. Photochemistry & Photobiology, 1999. 70(2): p. 191-8.
103. Wenczl, E., et al, (Pheo)Melanin Photosensitizes Uva-Induced DNA Damage in Cultured Human Melanocytes. Journal of Investigative Dermatology, 1998. 111(4): p. 678-82.
104. Kvam, E. and R Tyrrell, The Role of Melanin in the Induction of Oxidative DNA Base Damage by Ultraviolet a Irradiation of DNA or Melanoma Cells. Journal of Investigative Dermatology, 1999. 113(2): p. 209-213.

All publications listed above are incorporated by reference herein, as though individually incorporated by reference.

### BRIEF DESCRIPTION OF THE DRAWINGS

Embodiments, aspects, advantages, and features of the present invention will be set forth in part in the description that follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in these embodiments and their equivalents.

FIG. 1 depicts an embodiment of a block diagram illustrating an apparatus configured to provide melanoma related information.

FIG. 2 shows an embodiment of the low frequency EPR spectra obtained from differing amounts of eumelanin.

FIG. 3A shows a copy of a photograph of melanoma-bearing Xiphophorus hybrid, where the box shows the location of the tumor that was excised for measurement.

FIG. 3B shows low frequency EPR spectra of the excised tumor shown in FIG. 3A.

FIGS. 4A-B show an EPR imaging of Xiphophorus melanoma.

FIG. 5A shows a H&E stained section of melanoma from FIG. 4A.

FIG. 5B shows a H&E section of normal non-melanoma tissue.

FIGS. 6A-B show M. domestica (shaved) at months after a 2-stage DMBA application.

FIGS. 7A-C show H&E stained section of focal melanocytic hyperplasia and melanoma lesions in M. domestica treated with the 2-stage DMBA protocol.

FIGS. 8A-8B show in vivo EPR instrumentation for measurement of radical species in skin.

FIGS. 9A-B illustrate a surface coil resonator.

FIGS. 10A-B show a whole body resonator.

FIG. 11A shows a sectioning of embedded lesions perpendicular to the uppermost surface of the lesions.

FIG. 11B shows a sectioning of embedded lesions parallel to the surface of the lesions of FIG. 11A.

FIG. 12 shows an EPR spectrum of UV-induced melanin radical in murine skin using ex vivo UV illumination.

FIG. 13 shows a normalized action spectrum of UV-induced melanin radical in pigmented murine skin ex vivo together with the action spectrum of melanoma induction in Xiphophorus.

FIGS. 14A-B illustrate in vivo EPR instrumentation for measurement of UV induced radicals.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention. Other embodiments may be utilized and structural, logical, and electrical changes may be made without departing from the spirit and scope of the present invention. The various embodiments are not necessarily mutually exclusive, as some embodiments may be combined with one or more other embodiments to form new embodiments. The following detailed description is, therefore, not to be taken in a limiting sense.

In an embodiment, electron paramagnetic resonance spectroscopy is used to provide a signal from melanin to image a melanoma, to detect metastatic melanoma in a sentinel lymph node, to measure light penetration in melanocytes in skin, or combinations thereof. In various embodiments, EPR techniques may be applied in vivo including in vivo techniques to detect primary cutaneous melanotic lesions.

Electron Paramagnetic Resonance is a spectroscopic technique that sensitively and specifically detects free radicals. EPR is analogous to nuclear magnetic resonance (NMR), using a magnetic field and radiofrequency source to sensitively and specifically detect free radicals. In an EPR procedure, a sample is placed in a magnetic field and the absorption of applied radio frequency (RF) energy by unpaired electrons of free radicals is detected. The EPR technique has recently been extended by instrument development to allow in vivo spectroscopy and even (in favorable cases) imaging of free radicals. These techniques have been used in vitro and in vivo, and in studies of free radical biochemistry in skin tumor promotion. Importantly as a major advantage, EPR directly and non-invasively detects, quantifies, and identifies reactive free radical intermediates, as compared to product studies where the reactive intermediates must be inferred and the sample processed. In an embodiment, EPR may be used to detect free radicals such as melanin radicals and ascorbyl radical both in vitro and in vivo.

FIG. 1 depicts an embodiment of a block diagram illustrating an apparatus 100 configured to provide melanoma related information. Apparatus 100 includes an electron paramagnetic resonance spectroscopy unit 110 coupled to a analyzing unit 120 to analyze an electron paramagnetic resonance signal provided by the electron paramagnetic resonance spectroscopy unit 110 to provide melanoma related information. Analyzing unit 120 may be configured to image a melanoma, to detect metastatic melanoma in a sentinel lymph node, to measure light penetration in melanocytes in skin, or to provide combinations thereof. Analyzing unit 120 may include a database containing properties associated with a melanin signal to automatically assess the electron paramagnetic resonance signal. In an embodiment, electron paramagnetic resonance spectroscopy unit 110 may include a microwave bridge with an external loop electron paramagnetic resonance resonator. The microwave bridge may be configured to operate at 1 GHz. Apparatus 100 may include a UV irradiation source. The UV irradiation source may include a fiber optic device. Analysis unit 120 may be configured to assign a melanoma prevention factor (MPF) to a sunscreen.

Electron paramagnetic resonance spectroscopy unit 110 and analyzing unit 120 may be configured to provide an image of a melanoma at various depths from a skin surface. The image of a melanoma may include a depth of 0.5 mm to 0.10 mm from a skin surface. The image of a melanoma may include a depth of 0.10 mm or more from a skin surface. Electron paramagnetic resonance spectroscopy unit 110 and the analyzing unit 120 may be configured to determine the status of a sentinel lymph node with respect to metastatic melanoma. The determination of the status may be made in less than about 20 minutes. Apparatus 100 may be used to examine excised tissue or tissue in vivo.

Various embodiments may be used to image a melanoma using an EPR signal from melanin endogenous to the melanoma. In an embodiment, in vivo EPR techniques at 1 GHz frequencies and advanced surface and/or whole body coils may be used to provide an optimized combination of RF tissue penetration and sensitivity for studying free radicals in vivo. Such tissue penetration may be approximately 10 mm. Since melanin is already widely used optically as an endogenous contrast agent, an embodiment of the invention provides in vivo EPR techniques to detect and image even thick melanoma, using the inherent molecular contrast of melanin to allow rapid, noninvasive melanoma imaging, staging, and assessment of local lymph node involvement. In an embodiment, 1 GHz EPR spectroscopy with surface and whole body coils may be used to detect the melanin EPR signal in melanotic lesions. Both the premelanoma lesion focal melanocytic hyperplasia (FMH) and malignant melanoma may be observed, allowing for both early and later tumor detection.

According to an embodiment of the inventive subject matter hereof, EPR imaging may be used to image the depth of FMH and primary melanomas. Magnetic field gradients may be applied to allow spatial imaging of melanoma depth and margins, and correlate EPR images with histological measurement. According to an embodiment of the inventive subject matter hereof, EPR ‘virtual biopsy’ of lymph nodes or other tissue may be performed.

EPR imaging may allow 3-dimensional imaging of even thick melanomas, and can easily be linked to an optical imaging modality (e.g. dermoscopy or multispectral imaging) for coregistration and multimodal imaging. The molecularly based, high contrast imaging of melanoma for determination of thickness providing accurate initial staging of the disease can assist clinical choice of treatment options. Furthermore, it may allow accurate determination of melanoma morphology and extent in 3 dimensions, enabling resection margins to be accurately determined in a lesion-specific, rather than empirical manner. The detection of clusters of melanoma cells present in surrounding lymph nodes providing ‘virtual biopsy’ may also be very useful, as this is another powerful predictor. According to an example embodiment, tests of the methods hereof are tested on an appropriate animal model or using clinical human studies, where the utility and effectiveness of the methods are tested in human melanoma.

Tumor depth from 0.5 to 10 mm is the best staging and prognostic factor, while regional lymph node metastasis is also a major determinant of outcome, so determination of these factors is important. However, optical imaging techniques, though providing high contrast, are limited to a superficial depth range of 0.2 to 0.3 mm, and more penetrating imaging modalities, such as MRI, ultrasound, and near infrared (NIR) optical coherence tomography, have very low contrast properties for melanoma, as melanin is not well detected by these techniques. In an embodiment, EPR imaging may both detect and image melanoma at depths of 10 mm or greater, with high and molecularly specific contrast, due to its specific detection of melanin radicals.

Melanin comprises an average of about 1.5% wet weight of human melanoma tissues (with a 3:1 ratio of eumelanin to pheomelanin), and so represents a significant component of these tumors. Furthermore, melanin contains a large concentration of stabilized free radicals that are formed during its polymerization, so that EPR is an important technique for studying melanins. The EPR signal of melanin has been used to study photobiology in excised skin samples. EPR spectroscopy has also been used to study the melanin content of excised tissues. The potential for using the EPR signal of melanin to image melanoma was recognized some time ago. It is important to note that this was an extremely rudimentary experiment upon only the surface of an excised tumor, with somewhat questionable data. All these experiments were performed on isolated excised tissues, using ‘conventional’ X-band EPR techniques operating at approximately 9 GHz. However, the penetration of such microwave frequencies in ‘lossy’ aqueous samples is limited to approximately 0.1 mm, and this is why excised samples were used. Therefore, such instruments cannot either provide images of melanomas any deeper than approximately 0.1 mm, nor can it operate in situ. These and other technical details have prevented any useful development.

In the early 1990's several groups began using EPR spectrometers operating at lower frequencies to begin to study paramagnetic species in intact organisms, and this has developed into a very useful approach. Different groups have optimized instruments at different frequencies depending upon sample needs, but the L-band frequency range centered at approximately 1 GHz, developed by Walzcak and Swartz at Dartmouth and others has proven optimal for studies of tissues at depths up to about 10 mm. Several innovations, including advanced surface coil resonators, autotune-automatching microwave bridge-resonator combinations, and the development of magnetic field gradient coils and amplifiers for imaging, have made the instrumentation useful for in vivo and imaging studies. The improvements in sensitivity of these in vivo low frequency spectrometers have also extended the range of radical species that can be studied. In an embodiment, these developments in sensitivity have been sufficient to enable detection, and even imaging of the melanin in melanoma tumors in intact, non-excised samples.

Melanoma thickness is the single strongest parameter for staging and prognosis. Large studies have convincingly shown that tumor thickness is the single most powerful staging parameter and predictor of survival in localized melanoma disease. A continuous relationship between tumor thickness and survival has been shown, with the important range of thickness being 0.5 to 10 mm. This importance of depth is evident in a recent American Joint Commission on Cancer staging system. Conventional measurements of thickness prior to lesion excision are based upon determining lesion thickness from a biopsy, and so the entire tumor is not assessed for thickness. Although the lesion is palpated to choose a suitably thick biopsy site, it remains a point sample, and so may not accurately represent the global deepest penetration of a tumor. Likewise, after resection, the serial sectioning of the entire excised tumor and pathological examination of each section is currently the only way to ensure the maximum thickness in the entire tumor is measured. Yet, this can not usually be performed. In an embodiment, EPR imaging, as an imaging technique that globally and non-invasively may determine tumor thickness throughout the tumor, can offer significant advantages and may also be used to guide a point biopsy to the area of maximal tumor thickness. Thus, histopathology at the deepest and most invasive site may be determined. Additionally, in laterally invasive disease, a biopsy is guided to a similar site of maximal lateral invasion, or a resection margin determined directly according to the tumor, as opposed to margins that are more empirically derived.

Optical techniques are generally limited to superficial depth, while other techniques lack contrast. The majority of developing techniques for imaging and diagnosing melanoma have been based upon various optical techniques for both image acquisition and analysis, such as multi-wavelength analysis or artificial intelligence for diagnosis. However, a major limitation of these optical techniques is that they cannot image deeply into the skin, due to both light scattering and absorbance, especially in highly pigmented lesions. For example, dermoscopic, multispectral and confocal techniques are limited to maximum depths of 0.2 to 0.3 mm. Optical coherence tomography, using longer wavelength near IR light, has maximum depths of 1 mm but results in loss of melanin contrast.

This inability to detect and resolve non-superficial melanoma is a major disadvantage of optically-based techniques, as tumor thickness is the single most powerful predictor of survival in localized melanoma disease. The range of tumor thickness allowing this diagnostic prediction is in the range of the range 0.5 to 10 mm. Ultrasound techniques show some promise, although there is again a lack of contrast between melanoma and surrounding tissue, and under- and overestimates of thickness are common. This limits its primary usefulness to preoperative planning of diagnosed melanomas. MRI of melanoma lesions shows potential, with the development of high resolution surface coils, although its contrast specificity for melanoma remains low, and it is again primarily used for preoperative planning of pre-diagnosed melanomas. In an embodiment, EPR imaging, as a technique of melanoma imaging that has the ability to image deep into the skin to a depth of approximately 10 mm and that uses melanin as a high sensitivity endogenous contrast agent, may provide an advance in the field.

Existing developments in EPR technology may allow rapid clinical applications, at reasonable cost. Some considerations when developing new medical technologies are: a) the length of time to develop this nascent technology into a truly clinically-available application so that clinical trials can be performed, and b) whether this technology ultimately can be realized at a cost that can be justified by the benefits it brings. The NIH-funded in vivo EPR Center at Dartmouth has developed whole-body EPR magnets and instrumentation for clinical use and is currently performing clinical EPR trials such as trials of EPR oximetry. Overall, several of the key potential limitations to clinical and commercial development have been, or are being addressed.

Regional sentinel lymph node metastasis is an additional major determinant of outcome. The unfortunate propensity for melanoma to metastasize is primarily responsible for the mortality caused by this cancer. This metastasis occurs via the lymphatic drainage, and so local lymph nodes are usually primary initial sites for metastasis. Elective (total) lymph node dissection (ELND) in the local field is useful in staging, but does not significantly impact upon outcome in metastatic disease, and is associated with significant morbidity, except perhaps in selected subsets of patients. Recent efforts have focused upon minimally invasive identification of the first lymph node into which the melanoma drains—the ‘sentinel’ lymph node (SLN), followed by its excision and analysis. Although there has been some controversy, it has been broadly documented that this is a useful procedure, minimizing morbidity of ELND, while retaining the staging power of knowledge of metastasis and having been shown to especially be useful in melanomas with thickness greater than 1 mm. In thin melanomas (less than 1 mm) however, its overall cost-effectiveness is low, due to lower rates of metastasis in thin lesions. Overall, although controversial, SLN analysis shows significant promise. However, the intraoperative use of frozen section analysis of the SLN is not recommended because it is much less sensitive than conventional immunohistochemistry of fixed sections. Furthermore, the use of Positron Emission Tomography (PET) to detect and image metastasis only has a 14-22% detection rate. Even conventional post-operative sectioning has disadvantages, in that typically only a few sections are examined as total sectioning of every lymph node and pathological analysis can be inordinately expansive.

Because of these inherent disadvantages of current techniques, it can obviously be highly advantageous to have a sensitive technique to detect the presence of melanoma cells within the SLN at the time of operation, so as to guide surgical decisions at the time of surgery, as opposed to requiring subsequent procedures. Embodiments of EPR analysis of the SLN may allow a sensitive intraoperative determination of melanoma metastasis into the SLN, noninvasively so that subsequent pathology is not compromised. In an embodiment, evident lymph nodes may be analyzed in situ, without requiring dissection, in effect providing a ‘virtual biopsy’. In an embodiment, EPR detection of melanoma metastasis in lymph nodes may be performed such that the entire node is sampled at once, and then if desired, either imaged or excised.

In various embodiments, electron paramagnetic resonance spectroscopy may be used to detect metastatic melanoma in a sentinel lymph node. In addition to its visual properties, melanin present in melanocytes is paramagnetic, and so has properties that can be detected by various EPR embodiments. In an embodiment, these properties to very may be used sensitively to detect any melanoma metastasis in lymph nodes or other tissue, to allow accurate efficient and timely analysis of the metastatic status of this tissue. The sentinel lymph node (SLN) is the first node in the local lymphatic basin to which the tumor drains, and its status is thus uniquely and sensitively indicative of lymph node metastases. Accordingly SLN status is one of the most powerful predictors of the outcome of patients with stage 1 or II melanoma. The status of the SLN is also valuable in planning for melanoma patients, as both conventional and developing treatments are offered according to the cancer stage. SLN status is a major determinant of the cancer stage.

In an embodiment, EPR detection of melanoma metastasis in excised tissue may be performed. The EPR signal of melanin in any metastatic melanoma in the SNL, or indeed other tissue, can be detected on excised tissue. The status of an excised tissue may be accurately determined during the course of an operation, and the information given intraoperatively to the surgeon. The time taken would be less than about 20 minutes. The sensitivity may be very high, depending upon the microwave frequency band chosen for the EPR procedure. In an embodiment, specific properties of the EPR signal generated using EPR spectroscopy may be detected to unambiguously confirm that the signal is that of melanin. Such specific properties may include, but are not restricted to g value, linewidth and shape, power saturation properties, T1 and T2 of the EPR signal etc. The tissue is not physically changed by this process, so that it can be analyzed by conventional histopathology afterwards. Thus, the surgeon, knowing the status of the sentinel lymph node, could elect to not proceed with regional dissection if there is no overt disease, or, if the SNL is positive, continue with further dissection until the removed nodes are no longer positive. In an embodiment, an expert database of these properties may be constructed so that the EPR-based device automatically assesses the signal itself, without the need for an operator expert in EPR analysis, and gives the operator a readout with confidence intervals.

Depending on the microwave frequency used and the size of the tissue, it may be necessary to freeze the sample to prevent non-resonant absorption of microwave energy. Such knowledge is apparent to one skilled in the art. Thus a lymph node of mass 200 mg could be frozen and analyzed whole in an X-band spectrometer, and many other configurations are possible.

In an embodiment, EPR detection of melanoma metastasis in tissue in situ may be performed. Instead of excising the tissue, low-frequency EPR techniques may be used to assess the status of these tissues in situ. Although there may be slightly lower sensitivity than some detection protocols of excised tissues, this in situ procedure may be counterbalanced by the non-invasive nature of the procedure. The EPR detection scheme would be very similar, however, to a procedure excising the tissue. Other clinical implications and uses of the knowledge of lymph node status are also possible.

Various embodiments using EPR spectroscopy may be used for molecular assay of the UVA protective efficacy of sunscreens. Prevention by avoiding solar light and tanning booths, and use of sunscreen is central to public health programs. However, melanoma, unlike other skin cancers, is linked to longer wavelength UVA irradiation. The conventional SPF of a sunscreen relates to how well it protects against sun induced erythema, and therefore only measures UVB, not UVA. Yet, being able to measure a ‘UVA-SPF’ is very important in sunscreen design and testing. Some optical techniques have been described, but these have a number of disadvantages, both technically and in their conceptual development.

In an embodiment, electron paramagnetic resonance spectroscopy may be used to measure a UV induced radical signal from melanin to measure light penetration in melanocytes in skin. The effectiveness of a sunscreen may be determined to protect against melanoma based on the UV induced radical signal from melanin. The effectiveness of UVA sunscreens may be directly and accurately measured by its ability to reduce the UVA increase in melanin radical formation. A ‘melanoma protection factor’ or MPF mathematically calculated factor may be assigned to the sunscreens.

In addition to its optical properties, melanin contains a high concentration of stable free radicals formed by oxidation of its aromatic amino acid side-chains that may be detected by EPR techniques. Importantly, and central to its role in melanoma causation, it has also been shown in vitro that illumination of melanin by UV and blue light generates further meta-stable melanin radicals, causing oxygen consumption and also release of reactive oxidants such as superoxide (and hence hydrogen peroxide and potentially hydroxyl radical,) with essentially identical action spectra. Importantly, this suggests that these light-induced melanin radicals could be an appropriate surrogate measure for oxidant release into the melanocyte, and hence the potential of light to cause melanoma. The observation of oxidative DNA damage by UVA irradiation of melanin and precursors in vitro and in melanocytes bears witness to the importance of this process. These meta-stale light-induced melanin radicals have been observed by EPR in UV irradiated intact pigmented rabbit skin. This observation prompted a test as to whether an action spectrum of melanin radical formation could be generated in situ in the melanocytes of Xiphophorus skin, thereby gaining a deeper understanding of factors that result in the action spectrum of melanoma causation. As will be seen, the correlation between the two was in fact great, suggesting that oxidants formed in melanocytes by the action of UVA upon melanin playa central role in melanoma causation.

The measurement of UV induced radical signal of melanin to measure light penetration into the melanocytes in skin may be achieved in human skin using low frequency equipment and surface resonator. The received UV doses may be smaller than those required for other endpoints such as erythema, although extended illumination to test photostability is possible too. All skin types can be studied, and if required, the external loop can be centered on nevi (moles) to see how the sunscreen protects against UVA in these important sites too. Furthermore, the measurement is absolute, and hence free of potential bias that other visually scored tests, such as persistent pigment darkening (PPD), work upon. In addition to its use in humans, embodiments of the technique may also be applied to a wide range of animal models of melanoma, and the MPF of sunscreens calibrated against their ability to protect against melanoma, a major advance.

In an embodiment, EPR spectroscopy may be uniquely able to non-invasively determine and quantify these radical species in melanocytes in situ, in intact skin. In an embodiment, the response of non-excised, human skin may be determined, especially in melanocytic nevi prior to excision. Thus, direct comparisons between measurements made in models and humans may be possible. In an embodiment, electron paramagnetic resonance spectroscopy to measure a UV induced radical signal from melanin to measure light penetration in melanocytes in skin may be used to formulate a sunscreen having a high melanoma prevention factor.

Although the EPR apparatus is currently expensive, it is not exorbitant, and the manufacturers have experience in adapting the machinery to a specific protocol in EPR dosimetry of ionizing radiation. Volume savings from selling many of these e-scan machines have reduced the price considerably.

In various embodiments, electron paramagnetic resonance spectroscopy may be applied to animals and animal models to optimize in vivo EPR techniques to detect primary cutaneous melanotic lesions. Melanomas may be induced in the mammal Monodelphis domestica by topical 7,12 dimethyl(α)benzanthracene (DMBA) application, and 1 GHz EPR spectroscopy with surface and whole body coils may be used to detect the melanin EPR signal in melanotic lesions. Both the premelanoma lesion focal melanocytic hyperplasia (FMH) and malignant melanoma may be observed in this model, allowing determination of suitability for both early and later tumor detection. Using this melanoma model, magnetic field gradients may be applied to allow spatial imaging of melanoma depth and margins, and correlate EPR images with histological measurement. This use of EPR imaging may be conducted to image the depth of FMH and primary melanomas. In an embodiment, application of EPR to animals may be used to validate the EPR ‘virtual biopsy’ of lymph nodes. Known volumes of FMH lesions may be implanted post mortem at differing depths into tissues to establish the minimum detectable lesion sizes in deeper tissues.

The Monodelphis domestica melanoma model will allow testing of imaging and detection hypotheses. Although the Xiphophorus hybrid fish melanoma model possesses several advantages, and was used in initial method validation studies, the phylogenetic distance between this species and man is compounded by the very different skin structure, making this a suboptimal model for further development. A mammalian melanoma model that recapitulates many of the features of human skin and melanomas is required. The HGF/SF transgenic mouse model shows enormous promise in understanding melanoma causation, as it possesses human-like histopathology and benefits from murine models. However, HGF/SF mice do not express melanin as an albino FVB mouse background is used for their generation and propagation. This results in the formation of a melanotic melanomas (i.e. melanomas devoid of any melanin) that are not representative of human melanomas except in albinos. Their lack of melanin also precludes their imaging by EPR. Although future studies can benefit from establishing this transgenic model in a pigmented strain, the success of this approach is not guaranteed. Therefore, according to one example embodiment of the inventive subject matter hereof, it may be used and/or tested on a proven melanoma model in Monodelphis domestica, which possess several key advantages. Firstly, M. domestica is pigmented, and melanotic lesions in this model are darkly pigmented with melanin. Importantly, it is a model known to be susceptible to induction of the premelanoma lesion, focal melanocytic hyperplasia (FMH). FMH can be induced through treatment with either UVA, and/or UVB alone, but a much more rapid and efficient method is with topical 7,12 dimethyl(α)benzanthracene (DMBA) treatment. This produces multiple focal melanocytic hyperplasia lesions within 2 months, which progress to melanoma, and metastasize. Thus, this model will allow rapid production of both premelanoma FMH and malignant melanoma lesions, so that the application of EPR for the detection and imaging of these lesions can rapidly be achieved early in a scheduled research plan. M. domestica is a small laboratory animal (maximum grown weight 150-250 g) that is readily housed and bred and is used in a wide range of studies.

Melanin detection limits may be obtained with low frequency in vivo EPR techniques. In an embodiment, since melanoma tumors contain about 1.5% melanin, one use of the inventive subject matter hereof is to provide some estimate of the limits of detection of the in vivo low frequency EPR spectrometer, using purified eumelanin. Since many previous low frequency EPR spectrometers have been of relatively low overall sensitivity in comparison to the higher frequency conventional X-band machines operating at approximately 10 GHz, one goal is to make an improved sensitivity spectrometer and make certain that is has enough sensitivity. If only 30 mg of pure melanin can be detected, the method might be limited to detecting a minimum of about 2 grams of melanoma tissue, so it can be limited to quite large tumors. The eumelanin was from Sepia officinalis, a widely accepted standard for human eumelanin. Although this eumelanin does contain a certain amount of associated protein, this somewhat simplistic use of dry weight of this compound will actually underestimate the potential sensitivity.

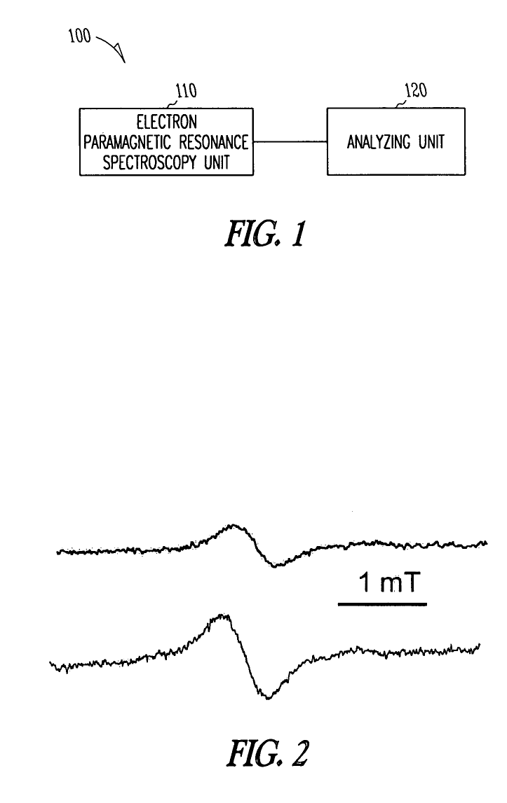

FIG. 2 shows the low frequency EPR spectra obtained from differing amounts of eumelanin, using the exact same surface coil resonator used for in vivo melanoma detection. The differing amounts are a) 0.25 mg and b) 0.5 mg melanin. Spectral acquisition time, scan time, is limited in this case to 21 seconds with modulation amplitude 0.4 mT and microwave power 20 mW. Although more experiment time can be used to improve signal:noise ratios by averaging large numbers of spectra, it was desired to replicate a realistic intraoperative surgical application, such as an SLN analysis. It can be seen from FIG. 2 that the detection limits, even under these stringent conditions, were about 0.1 mg of eumelanin, implying a realistic sensitivity of better than 10 mg of melanoma tissue (on average) with better sensitivity available if needed. Thus, it appears that potentially even small melanomas can be accurately and rapidly detected by this technique, even under stringent conditions.

In an embodiment, detection limits may be obtained in an animal model of melanoma. According to one example embodiment, the sensitivity limits were experimentally examined in an authentic melanoma. Although the sensitivity of this technique was obviously high for isolated melanin, and although one would not predict any major difference in the EPR signal or intensity of isolated, as opposed to in situ melanin, the sensitivity limits in an authentic melanoma may be experimentally examined. For this preliminary work, a well characterized fish model of melanoma in Xiphophorus hybrids was used, in which UV light induces locally aggressively infiltrating melanotic melanomas. Although there is obviously some phylogenetic distance between fish and humans, they are a useful model. Many features of human melanoma pathology are recapitulated in this model. Fish contain predominantly eumelanin and the relatively small size of melanomas apparent in these small fish is readily apparent by observing gross anatomy providing a further test of sensitivity.

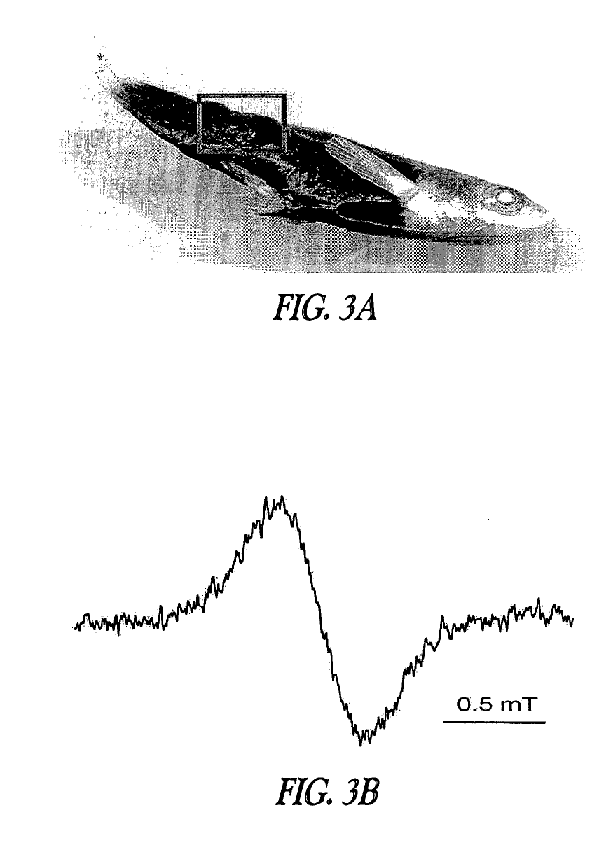

FIG. 3A shows a copy of a photograph of melanoma-bearing Xiphophorus hybrid, where the box shows the location of the tumor that was excised for measurement.

The melanoma on the flank of the fish in FIG. 3A of size 2.5×2.5×1 mm, wet weight approximately 7 mg, was dissected out of the euthanized animal and placed in the loop of a low frequency surface coil EPR resonator. Dissection was necessary as the spectrometer is so sensitive that it can detect the melanin in the thin normal melanocyte layer in the skin of these fish. FIG. 3B shows a low frequency EPR spectra of the excised tumor shown in FIG. 3A, obtained using the surface coil as in FIG. 2. FIG. 3B shows the EPR spectra recorded from the melanin in this tumor using an acquisition time of only 30 seconds with modulation amplitude of 0.4 mT and microwave power of 20 mW. Non-melanotic skin of the same wet weight gave no detectable signal (not shown). It can be seen that the melanin in this small tumor was readily detected with a signal:noise ratio of over 8:1, even under these stringent conditions. Thus, the sensitivity required for experiments relating to a ‘virtual biopsy’ capability is already achievable. Improvements may be possible with improvements both instrumentally and by allowing more acquisition time for signal averaging.

In an embodiment, EPR imaging in an animal model of melanoma may be conducted. Above, it is established that the detection sensitivity of the low frequency EPR system with the surface coil may be high and that the approach may well able to record a spectrum of the melanin in a melanoma in the Xiphophorus tumor model. A good signal:noise ratio was readily obtainable. It was also determined that it was possible to use the imaging capabilities of the Bruker E540 imaging spectrometer to image melanomas in the Xiphophorus model. It is important to note that these images were obtained using the volume resonator due to the gradient coils for the surface coil being unavailable at that time. This whole-body, volume resonator has a diameter of 3.5 cm and a length of 4.2 cm, and so the filling factor (essentially the ratio between sample volume and the active volume of the resonator) for this small sample was very poor. Despite this technical disadvantage, EPR imaging was able to successfully image melanoma in this model.

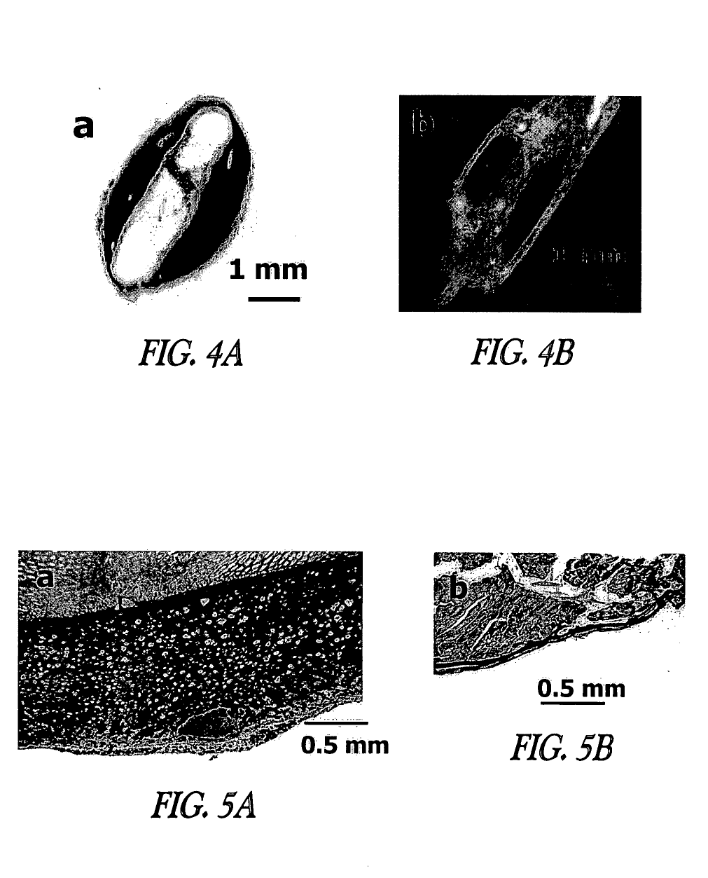

Shown in FIGS. 4A, 4B is an embodiment of EPR imaging of Xiphophorus melanoma. FIG. 4A shows a paraffin embedded (wax-embedded) transverse section from a fish bearing two highly pigmented melanoma tumors, one on each flank. FIG. 4B shows the appropriate slice from the EPR image of melanin distribution in the intact fish sample. FIG. 4B shows an EPR image of melanin signal in the same section with slice thickness of 0.25 mm. The adjacent bar denotes the image intensity scale. The complete series of 49 EPR image slices of the entire fish took 25 minutes to acquire. It can be seen that even in this somewhat suboptimal experimental configuration and performed in a preliminary fashion, relatively small melanomas in this fish model can be accurately imaged by using the EPR signal of their endogenous melanin. The close correlation between the gross optically apparent tumor morphology and the EPR image is readily seen, as is the accuracy of tumor thickness estimation.

FIGS. 5A and 5B provide histology of Xiphophorus melanoma. H&E stained sections of this melanoma (FIG. 5A) and normal tissue (FIG. 5B) are presented showing the dense melanin pigmentation of the thick tumor tissue that can be seen invading the underlying muscle tissue. In comparison, the dermal melanocytes in normal skin form the usual thin superficial layer. Thus, there has been demonstrated the potential for EPR imaging of even small melanomas, using the whole body resonator. Signal:noise ratios can be further improved by use of the whole body resonator for the larger lesions of M. domestica, or by use of the surface coil.

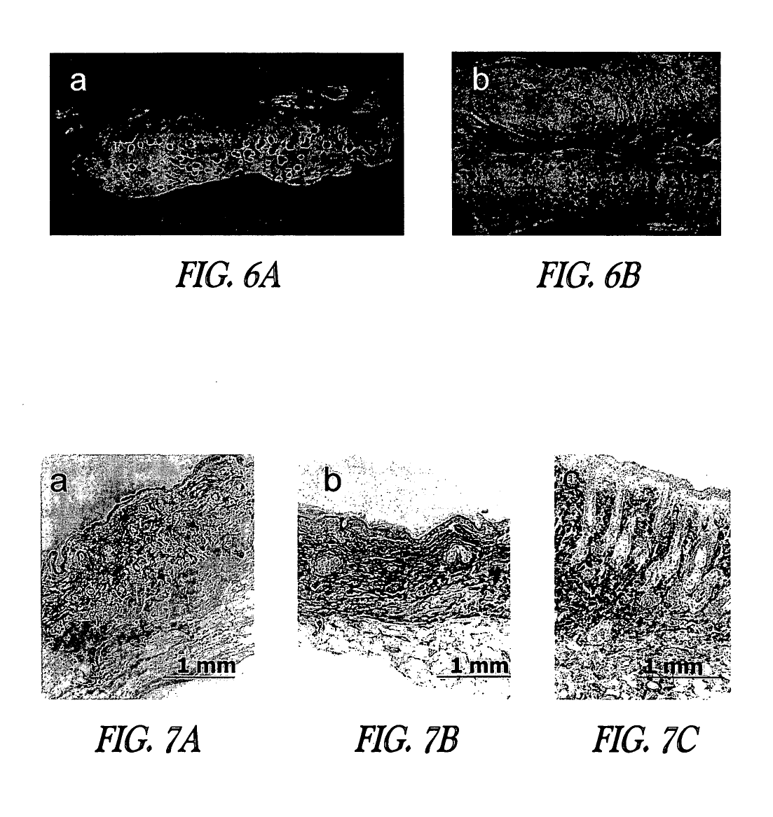

The M. domestica model of melanoma possesses all the requirements and several advantages for study of an embodiment. Initial studies into the use of DMBA as an initiating agent used a 2-stage DMBA application protocol of 100 μl of 0.5% DMBA applied to the back, with a repeat treatment 3 days later. This resulted in extensive FMH lesions in every animal of approximately 20 to 50 lesions within 2 months that is greater and more rapid than the prevalence or multiplicity of FMH in UV treated animals. FIGS. 6A and 6B show M. domestica (shaved) at months after the 2-stage DMBA application. FIGS. 7A-C show H&E stained section of FMH and melanoma lesions in M. domestica treated with the 2-stage DMBA protocol. Representative sections of such FMH lesions (premelanoma lesion in this animal model) are shown in FIGS. 7A and 7B, showing the dense concentrations of highly melanotic melanocytes at depths of 1-2 mm. A proportion of these FMH lesions also spontaneously progressed into malignant melanoma and also metastatic malignant melanoma. A more invasive melanoma is shown in FIG. 7C, showing deeper invasion, a dermal melanoma lesion showing melanocyte invasion into underlying tissues not see in FMH. Although the location of these FMH and melanoma lesions is primarily dermal, as opposed to epidermal in humans, the greater depths of these lesions allow a more rigorous test of the sensitivity and depth penetration of the technique. So for these particular studies, this is actually an advantage. It can be seen that these lesions represent excellent model for the development of EPR imaging studies, based on their pigmentation, rapid induction, and progression from FMH to melanoma.

Thus, according to one example embodiment, in vivo EPR techniques, according to example embodiments of the inventive subject matter, can detect and image even thick melanoma, using the inherent molecular contrast of melanin to allow rapid, noninvasive melanoma imaging, staging, and assessment of local lymph node involvement.

According to one example embodiment, in vivo EPR techniques are optimized to detect primary cutaneous melanotic lesions. In this embodiment, an experiment is conducted to induce premelanoma FMH and primary malignant melanoma lesions in M. domestica and use in vivo EPR with an advanced autotune/automatch surface coil resonator to detect the melanin radical signal in these lesions during their growth. The experiment may also be conducted to determine sensitivity thresholds as a function of systematic optimization of instrumental and experimental parameters.

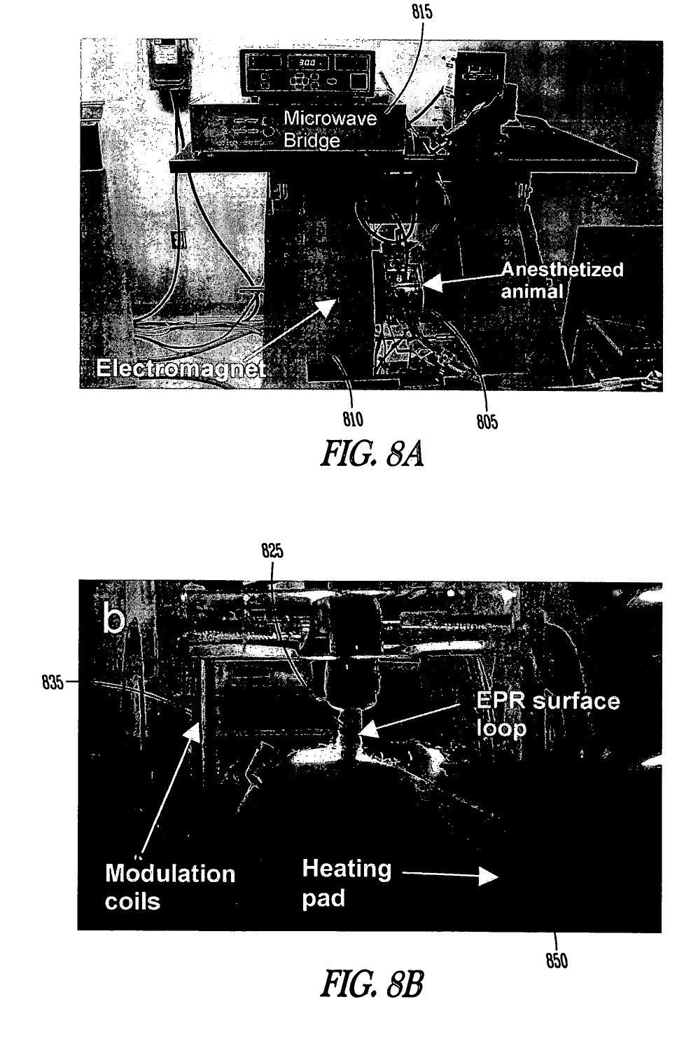

Electron Paramagnetic Resonance is a spectroscopic technique that sensitively and specifically detects free radicals, such as melanin radicals and ascorbyl radical both in vitro and in vivo. The sample is placed in a magnetic field and the absorption of applied radio frequency (RF) energy by unpaired electrons of free radicals is detected. EPR directly and non-invasively detects, quantifies, and identifies reactive free radical intermediates, as compared to product studies where the reactive intermediates must be inferred and the sample processed. To assist in visualizing the unique configuration of in vivo EPR instruments, FIGS. 8A and 8B show in vivo EPR instrumentation for measurement of radical species in skin. In an embodiment, the measurement of radical species in skin may be a result of UV irradiation for measuring UV-induced radicals in skin in vivo. Such a configuration may be used to examine the role of UVA-induced radicals in melanoma causation. FIG. 8A shows an overview of experimental setup using a microwave bridge 815 showing positioning of an animal sample 805 in an air gap of an electromagnet 810. FIG. 8B depicts a close up (from rear) showing animal 805 in position under the external loop surface coil resonator 825. FIG. 8B shows modulation coils 835, an EPR surface loop 825, and a heating pad 850 on which animal sample 805 may be placed. Salient features of in vivo EPR instrumentation for various embodiments are apparent.

An embodiment of a specific EPR methodology may be applied for in vivo studies of melanin radicals in FMH and melanoma induced in M. domestica. Shaved M. domestica is anesthetized using isoflurane, administered for induction in a suitable box, and for maintenance via a nose cone. To maintain rectal temperature at 33° C. the animal is placed on a thermostatically controlled heating pad for external-loop surface coil experiments. For whole body volume resonator use, the water cooling for the gradient coils is maintained at 33° C. A Bruker Elexsys spectrometer is operated at L-band frequencies, approximately 1.1 GHz, using a highly specialized and sensitive automatching/autotuning bridge. The static magnetic field may be provided using a 4″ air gap electromagnet, operating at approximately 40 mT (400 G) for resonance with a thermostatic hall effect probe controlling the field. Magnetic field modulation may be applied using calibrated field modulation coils that are specially designed to minimize microphonics. These are inbuilt for the whole body resonator, separate for the surface coil. Spectra are recorded using multiple-scan averaging techniques, as signal averaging is useful in in vivo EPR. Although numerically equivalent, a long-scan time single scan is susceptible to slight movement artifacts, whereas in multiple scans, any such movement is averaged to low levels. Two different resonator configurations are tested for their sensitivity and utility.

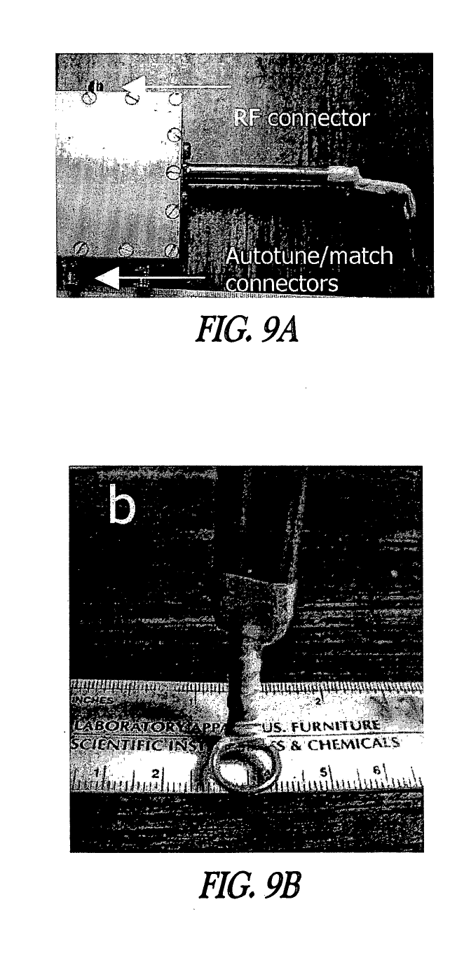

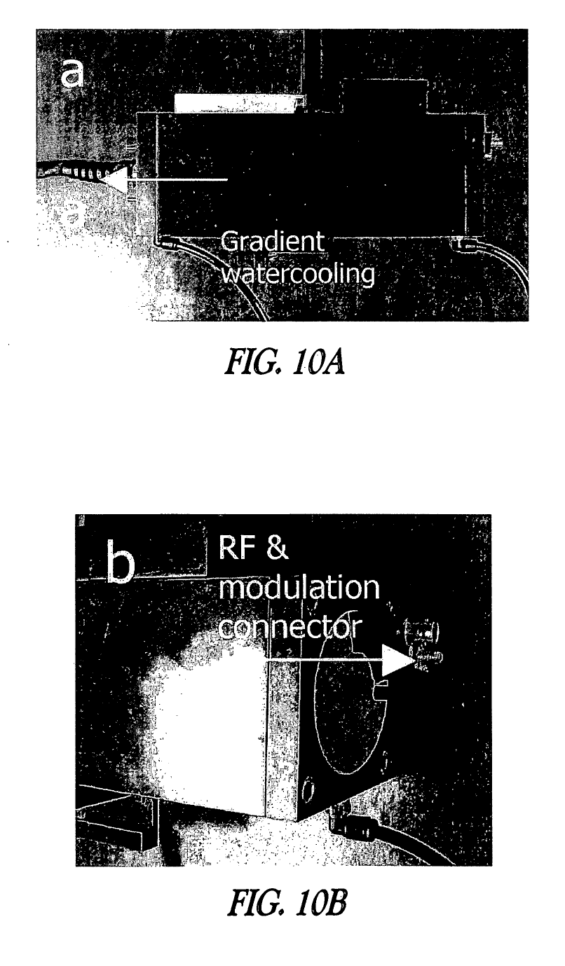

In an embodiment, an external loop surface coil resonator may be used. FIGS. 9A and 9B illustrate a surface coil resonator. FIG. 9A depicts the entire resonator showing RF and autotune/automatch connections. FIG. 9B illustrates a zoom of the surface coil, showing clear optical access of the sample probed beneath the loop. The external loop surface coil provides a very convenient experimental setup, as the loop simply needs to be placed directly above the lesion of interest (gently to prevent occlusion of blood flow to skin). The microwave B₁ field from such resonators has been imaged, and provides a useful region of suitable homogeneity of approximate diameter of the loop, with a depth of penetration of about 8-10 mm. Any small inhomogeneities in B₁ can be mathematically corrected for in the image processing process. This resonator requires the use of separate water-cooled anti-Helmholtz gradient coils for imaging, these have custom made in-house to provide a spherical area of high gradient linearity of diameter 1 cm. The primary advantages of this resonator are the ability to localize individual lesions physically and the high sensitivity in this smaller volume. Furthermore, the nature of the loop means that the visual monitoring of the lesion in situ in the resonator is readily performed. This allows visual confirmation that the lesion imaged is the one desired. In addition, this can allow the simultaneous use of dermoscopic and/or or multispectral imaging techniques for coregistration and even multimodal approaches.

In an embodiment, a whole body volume resonator may be used. FIGS. 10A and 10B show a whole body resonator. FIG. 10A depicts the entire resonator showing gradient power and cooling connections. FIG. 10B shows a zoom of sample access ports with RF and modulation connectors. This a specialized imaging resonator that contains built-in water-cooled gradient coils that provide continuous gradients in three orthogonal axes of up to 0.6 T m⁻¹. The microwave resonator is a specialized birdcage structure with a region of maximal axial RF homogeneity of 3.4 cm diameter across the sample and a sensitive length of approximately 3.5 cm. Data obtained using paramagnetic point samples indicates that the homogeneity of sensitivity (the combination of microwave field and magnetic modulation homogeneity) across the diameter of the resonator in this volume is within 25%, while that along the length is within 30% in the 3.5 cm length (maximum intensity in the center). Thus, this resonator is optimized for imaging large volumes, such as the entire FMH containing back of an animal. This resonator, however, does not allow optical access to the sample during imaging, so that coregistration is not simple. Also, large animals (age>6 months) will not fit into the resonator, so that larger animals can only be imaged with this resonator after sacrifice and some gross dissection, although this is performed routinely.

An embodiment of a methodology for optimization of melanin radical EPR signals in vivo may be performed. Due to the complex interactions of the RF and magnetic field modulation fields, microphonics can have complex effects on the noise floor, especially at the larger modulation amplitudes required to maximize sensitivity of detection of the somewhat broad (0.4 mT) linewidths of melanin. As a result, it is necessary to optimize the combination of incident microwave RF power, modulation amplitude, and modulation frequency to allow maximal RF and modulation amplitude, while minimizing microphonics. The automated multidimensional experiments possible with a Bruker Elexsys console allow a rapid ‘search’ of this multidimensional ‘noise floor space’ and greatly facilitate initial optimization. These multidimensional experiments may be automatically analyzed for signal height and signal:noise ratio. The experiments use the dimensions of incident microwave power (40, 20, 10, 5 and 2.5 mW) modulation amplitude (0.6, 0.5, 0.4, 0.3, 0.2 0.1 mT) and modulation frequency (100, 75, 50 and 25 kHz) requiring 120 different conditions to be initially tested. Since each scan is expected to take no more than 1 minute, though more like 20-40 seconds, this overall time (40-120 minutes) is within acceptable limits for an anesthetized animal preparation. Once a suite of 5-10 most favorable conditions is identified, these may be automatically programmed for rapid optimization in every subsequent experiment. However, where rigid quantitative comparison is required (for example comparing the EPR signal as a function of lesion volume or depth etc.), it is important to use identical instrument parameters. Thus, the signal from each of this ‘suite’ of 5-10 most favorable conditions is recorded and stored, allowing comparisons of the EPR signals between samples at each of these parameter combinations.

An example methodology for induction of FMH and melanoma in M. domestica is provided in the following. Treatments with 100 μl of 0.5% DMBA 3 days apart lead to the rapid development of FMH at high prevalence and multiplicity per animal, within 2 months. This protocol is used to develop FMH at high multiplicity. Male animals (2-3 months old) may be obtained from the Southwest Foundation for Biomedical Research, housed, and fed as previously detailed. They may be anesthetized with isoflurane and dorsal hair removed with clippers. Groups of animals may be treated with 2 treatments of 100 μl of 0.5% DMBA in ethanol, 3 days apart. These animals may be shaved weekly with a Remington microscreen shaver and FMH and cutaneous malignant melanoma prevalence and multiplicity noted. The back of each animal may be digitally photographed at 3 megapixel resolution and data archived for any retrospective analysis as may be required. These groups of animals may then have their lesions used for EPR studies as detailed subsequently. Experience shows, in some cases, that pigmented lesions that are not raised are usually found to be FHM upon histological examination, whereas those pigmented lesions that are raised (i.e. vertically invasive) are usually found to be melanoma on histology. Thus, all imaged lesions are so classified initially, but confirmed by histological analysis.

Experimental EPR detection and optimization of both FMH and primary malignant melanoma may be conducted. Two groups of animals may be treated with the DMBA protocol. The general in vivo EPR detection methodology with respect to previously described specific EPR methodology for in vivo studies of melanin radicals in FMH and melanoma induced in M. domestica may be used to detect the melanin EPR signal. Both the external loop surface coil and the whole body volume resonators may be used. The sample access size may preclude measurement of animals greater than approximately 6 months. So, the sample imaged may be the entire back of the animal after the gross dissection necessary before lesions are removed for histological and morphometric analysis. The methodology for optimization of melanin radical EPR signals in vivo discussed previously may be applied in each of these animals. Should it be apparent after at least half the group has been analyzed that use of the limited suite of 5-10 most favorable conditions covers all useable options, then the suite will be done. The animal will then be euthanized. The studied lesion(s) may be photographed, then carefully excised, and processed for morphometric and histological analysis. The optimized EPR signal intensity may then be correlated with lesion volume and depth.

Two groups of animals may be used. Group a may be categorized for EPR detection of early FMH lesions. A group of 10 animals may be used, as the high prevalence and multiplicity of FMH will allow study of at least one lesion in each animal. The animals may be studied at 2-3 months after DMBA treatment.

Group b may be categorized for EPR detection of malignant melanoma and late FMH lesions. A group of 20 animals may be used, as there is a lower prevalence of malignant melanoma, and not all animals are expected to progress to this end point. The animals may be continuously monitored and animals demonstrating primary malignant melanoma by gross examination and palpation (approximately 3-6 months after DMBA treatment) may be studied. At 9 months all non-primary malignant melanoma-bearing animals may be studied and euthanized with melanoma confirmed histologically.

Data analysis and correlation of EPR detection with lesion dimensions and histopathology may be conducted. Each EPR studied lesion may be excised, formalin fixed, grossly sectioned to a margin of 0.5 mm of normal tissue at the lateral margins of the lesion, and paraffin embedded using standard procedures. Transverse 10 micron thick sections may be made in triplicate every 0.5 mm across the diameter of the lesion. The area of melanotic tissue and the depth to which it extends may be determined using an Olympus BH2 upright microscope interfaced with the quantitative image analysis programs ImagePro Plus and Metamorph. The lesion volume may be then be determined by interpolating area between these transverse sections as a function of their section depth using the computer software. Maximal, median, and mean lesion depth may be determined. The optimized EPR signal intensity may then be correlated with lesion volume and depth to determine what the smallest detectable lesion is and also to determine how lesion depth affects signal intensity per unit lesion volume.

With respect to lesion multiplicity, it is anticipated that FMH is induced at high prevalence and multiplicity by the 2-stage DMBA protocol. Should lesion prevalence be unexpectedly high, so as to preclude the discrete lesions seen in the M. domestica model of melanoma discussed previously, group a will be repeated with half the DMBA concentration before progressing to group b. With respect to lesion sectioning, for very small diameter lesions (less than 2 mm in any dimension) the step of 0.5 mm between sections detailed above is too large for accurate volume determination, and steps of 0.25 mm may be used. Statistical analysis may be applied. Linear regression (Sigma Stat 3.0) analysis may be used to study EPR signal intensity as a function of lesion volume, lesion mean, median and maximal thickness, and the ratio of volume to thickness. As all these are ratio variables, linear regression is well justified. Although multiple regression analysis (by hierarchical stepwise regression) may also be performed, it is suspected that the use of relatively simple and robust analysis at this initial stage provides guides as to which variables are introduced in the hierarchical stepwise regression analysis. There is expected a high positive correlation coefficient between lesion volume and EPR signal intensity, simply as more melanin is present in a larger lesion. Also anticipated is a moderate correlation between signal intensity and ratio of depth to volume, as deeper melanin will have slightly less signal per amount than more superficial melanin, as there is some limitation of penetration of RF to deeper tissues.

At the conclusion of these experiments, the relevant EPR parameters may be optimized for in vivo measurements of melanin in melanotic lesions. Furthermore, these experiments may provide an understanding of the interplay between signal intensity, lesion volume, and lesion depth, so that one is able to accurately estimate the minimum detectable sizes of lesions at different depths, of particular relevance to the feasibility to EPR virtual biopsy of lymph nodes.

According to another example embodiment, EPR imaging may be used to image the depth of FMH and primary melanomas. In an embodiment, experiments may be performed to EPR image FMH and primary melanoma lesions, using both surface coil and whole body resonators, and to correlate the EPR images with histopathological assessment of lesion morphology and dimensions. The experiment may also be performed to critically evaluate the degree of correlation between EPR and morphometric analysis.

The field of EPR imaging, although well established by the early 1990's has undergone considerable recent progress for biomedical applications, as is exemplified by work of several groups worldwide. As with MRI, the spatial localization of spins is encoded by the application of magnetic field gradients, although the pulsed gradient sequences used in MRI are not generally applicable due to the much shorter relaxation times of electronic, as opposed to nuclear spins. Thus, images are obtained by obtaining projections over a swept range of magnetic field gradient angles and using filtered backprojection to produce the final image. The magnetic field gradients required are produced by anti-Helmholtz and/or figure of eight coils as described by Eaton (See, Eaton, G., Eaton, S. & Ohno, K. Epr Imaging and in Vivo Epr, CRC press, Boca Raton, 1991). High currents (2 to 20 amps) and continuous duty operation cycles usually necessitate air or water cooling. The maximal theoretical resolution is a function of EPR linewidth, attainable gradient strength, and the number of projections, although signal:noise ratios are also a factor.

An example of a specific EPR imaging methodology for M. domestica FMH and melanoma is provided in the following example. A Bruker E540 imaging EPR spectrometer possesses advanced software control of all image acquisition, processing and image analysis routines through proprietary XEpr and XView software. The calculated resolution as a function of linewidth, gradient strength, and number of projections is automatically calculated. The magnetic field gradient projections required for any given image are automatically determined and achieved through a dedicated hardware gradient controller and 3 individual current amplifiers for X, Y, and Z gradient coils capable of continuously supplying 20 amps at 35 volts in each axis. A whole body, volume imaging resonator contains water-cooled gradient coils that provide gradients in three orthogonal axes of up to 0.6 T m⁻¹. The in-house water cooled gradient coils for an external loop surface coil resonator are capable of gradients in excess of 0.16 T m⁻¹. An imaging protocol may use the optimized signal parameters from the methodology for optimization of melanin radical EPR signals in vivo previously discussed. After acquisition of an imaging dataset, the projections may be deconvoluted using a non-gradient EPR spectrum and filtered backprojection performed. Identical deconvolution parameters may be used to allow meaningful comparisons between gradient strengths.

An example experimental EPR imaging of both FMH and primary malignant melanoma may be performed. Two groups of animals may be treated with a DMBA protocol and the melanin EPR signal in the resultant melanotic lesions may be imaged. Both the external loop surface coil and the whole body volume resonators may be used to determine which provides optimal images. The surface coil experiments may be performed first, followed by the whole body resonator. Animals greater than age 6 months may not fit in the whole body resonator, and so the excised back of these animals may be imaged in this resonator after their in vivo study with the surface coil. The experiments may initially evaluate the use of 4 different gradient strengths, equivalent to 1, 2, 3 and 4 times the intrinsic linewidth per cm, (i.e. 0.4, 0.8. 1.2 and 1.6 mT cm⁻¹) to determine the optimal resolution achievable within useful signal:noise. As an example, the EPR image in FIG. 4B was obtained with gradient strengths of 0.8 mT cm⁻¹ with the resolution achieved being approximately 0.2 mm. The sample access ports of the whole body volume resonator may preclude measurement of animals greater than approximately 6 months of age, and so the sample imaged in this case may be the entire back of the animal after the gross dissection necessary before lesions are removed for histological and morphometric analysis. Before EPR imaging of a lesion it is marked at the margins with permanent marker, and then after imaging marked with a surgical ink tattoo to allow co-registration of the EPR image with the histopathological and morphological analysis. The animal may then be euthanized. Lesions may be photographed, excised, and processed for analysis as discussed previously for data analysis and correlation of EPR detection with lesion dimensions and histopathology.

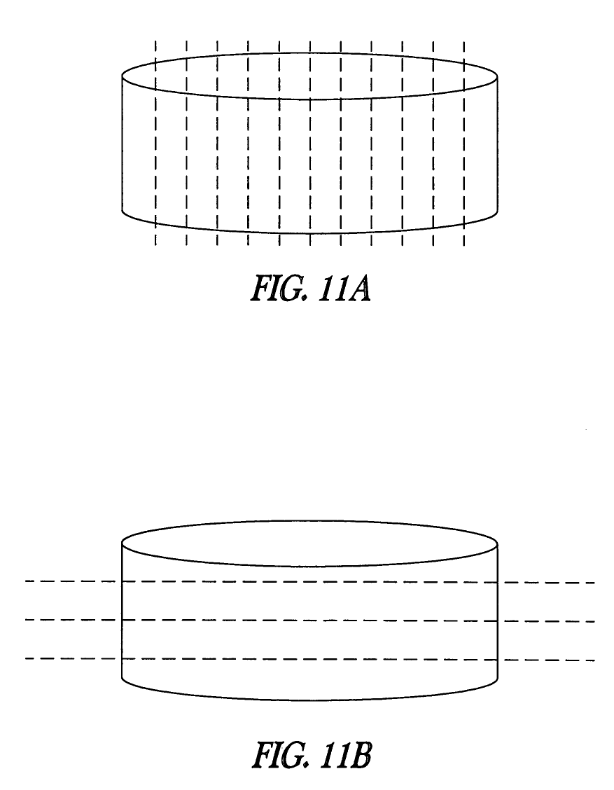

The two groups of animals may be organized as group a and group b. Group a may be categorized as EPR imaging of early FMH lesions. A group of 20 animals may be used. In order to fully assess the correlation between EPR imaging and histological morphology, two sectioning protocols are necessary. The lesions are embedded with half of the lesions sectioned perpendicular to the uppermost surface of the lesion, i.e. perpendicular to skin surface, as shown in FIG. 11A, to obtain accurate measures of thickness through the lesion, accurate cross-sections, for correlations of depth with EPR images. Half of the lesions are sectioned parallel to the surface of the lesion, i.e. parallel to skin surface, as shown in FIG. 11B, as to obtain accurate cross-sectional morphologies of the lesion in this dimension for correlation of cross-sectional area and lateral margins. The EPR images may be ‘sectioned’ by software interrogation in any axis desired. The animals may be studied at 2-3 months after DMBA treatment, the optimal time for early FMH.

Group b may be categorized as EPR imaging of malignant melanoma and late FMH lesions. A group of 20 animals may be used, as there is a lower prevalence of malignant melanoma, and not all animals are expected to progress to this end point. Depending upon final prevalence, perpendicular sectioning, as in FIG. 11A, may be used if less than 5 melanoma lesions are finally obtained, apportioning additional lesions to parallel sectioning, as in FIG. 11B, and then equally to parallel and perpendicular sectioning. This is done, as thickness is the single most important parameter, and so is focused upon firstly.

Data analysis and correlation of EPR imaging with lesion morphology and histopathology may then be conducted. As detailed above, all lesions that have been EPR imaged will have been marked with a surgical ink tattoo, and then photographed to allow co-registration of the EPR image with the histopathological and morphological analysis. The lesions may then be excised, formalin fixed, grossly sectioned to a margin of 0.5 mm of normal tissue at the lateral margins of the lesion, and paraffin embedded using standard procedures. The lesions may be physically sectioned in two different planes as detailed above with respect to FIGS. 11A-B.

The morphology of the lesions may then be determined using the same protocol and software as previously discussed with respect to data analysis and correlation of EPR detection with lesion dimensions and histopathology. Since the thickness of melanoma lesions is so central to the clinical outcomes observed, a critical evaluation of the cross sectional area and thickness of melanotic lesions in histological sections obtained perpendicular to the lesion, as in FIG. 11A, may be performed. These values may be compared to the relevant EPR image slices, using the ImagePro Plus and Metamorph software. The margins of the lesions in histological sections may be determined both manually, and also automatically by the software, and the area of each integrated areas and thicknesses may be obtained. The integrated areas of the EPR image slices and the lesion thickness may also be calculated. Linear regression may be then used to correlate these values from histological and EPR images, from each set of paired image slices, to gain a quantitative insight into the correlation between EPR and histology data. Objective examination of the morphology of the paired image slices may be used to assess how the various features apparent by visual examination (lesion margin shape, extent of infiltration into normal tissue, extent of melanin pigmentation) are correlated. Each slice may be coded and then be divided into a 5×5 grid of sub-sections, and each matching pair of subsections then randomized so as to blind an evaluator. The correlation between the matched pair of subsections for EPR and histology may be categorized for margin shape, infiltration, and pigmentation. Each may receive a score from 1 to 9 (1 being no apparent correlation, 5 being close correlation in half the sub-section field, and 9 being apparently identical throughout the subsection field). The correlation may then be numerically analyzed. Half of the EPR imaged lesions also physically sectioned parallel to the lesion (skin) surface as in FIG. 11B may be analyzed, in order to better correlate the lateral margins of the EPR and histological sections. Identical analytical procedures are used on the perpendicular and the parallel sectioned slices, again combining analysis of numerical values of lesion area with objective determinations of lesion shape, infiltration, and pigmentation as described above.

With respect to numeric analysis of lesion morphometry, linear regression may be used to compare the numerical values of EPR image and histological lesion area and thickness (all ratio variables) in each pair of sections. A high positive correlation coefficient between the values obtained by EPR and histological analysis is expected. The extent of correlation between EPR and histology images may also be studied as a function of the applied magnetic field gradient strength in order to determine if correlation is maximized by any one particular gradient strength. It is anticipated that, as long as an adequate signal:noise ratio is obtained, the higher the gradient strength, the better the image resolution and the better the correlation between EPR and histological analysis. Multiple regression analysis may also be used to allow inclusion of the average signal:noise ratio of the EPR projections as a variable in the analysis of correlation and gradient strength, as it is likely that that higher gradients will result in lower signal:noise ratios. Multiple regression analysis may be realized by hierarchical stepwise regression and by Sigma Stat 3.0 software. EPR projections may be automatically calculable using the Xepr software.

With respect to objective analysis of morphology, infiltration, and pigmentation, the variables in this analysis are derived from scored scales and as such are ordinal variables, although the relatively large scale range and relatively linear assessment will allow analysis as interval variables. The correlation between EPR and histology image variables may again be analyzed as a function of applied magnetic field strength and the average signal:noise ratio of the EPR projections. Multiple regression analysis may be used including multiple regression analysis by hierarchical stepwise regression.

With respect to gradient strength optimization, should it be apparent in early images that image quality increases with magnetic field gradient strengths in the preliminary range of up to 0.16 mT cm⁻¹, values of 0.2 and 0.24 mT cm⁻¹ may be further added with analyze of the corresponding images. The preliminary data in the Xiphophorus model previously described with respect to EPR imaging in an animal model of melanoma indicates these vales represent useful maxima, beyond which lowered signal:noise ratios negate the improved spatial resolution. These findings may be confirmed experimentally in the Monodelphis model.

With respect to analysis of resonator suitability, after there is a good understanding of how the different experimental and image-derived variables are correlated, a sub-set of the most informative images may be analyzed as a function of the type of resonator used to determine which may have the most optimal features for clinical studies. Should there be no significant advantages in image quality, one may focus future developments upon the surface coil resonator, as this allows easy placement over the lesion of interest and also allows optical coregistration techniques via the free space in the middle of the loop.

At the conclusion of this series of experiments, there may be a rigorous and quantitative analysis of just how well EPR imaging may be able to determine the overall morphology of FMH and melanoma lesions, using histology as the ‘gold standard’. There may also be knowledge available as to how the magnetic field gradient strengths and resonator types affect the quality of the EPR images.

According to another example embodiment of the inventive subject matter, EPR ‘virtual biopsy’ of lymph nodes or other tissue may be validated. According to this embodiment, experiments may be provided to implant known small volumes of FMH lesions post mortem into tissue, and use a surface coil resonator to determine minimal detectable volumes of melanoma.

As all techniques have been previously described, these example sections are referred to for brevity. Unimaged excess FMH tissue may be harvested from sections of groups of animals for induction of FMH and melanoma in M. domestica to detect primary cutaneous melanotic lesions and from sections of groups of animals to image the depth of FMH and primary melanomas, as the high lesion multiplicity will mean not all lesions can be imaged. This tissue may be snap frozen and stored in liquid nitrogen. When needed, appropriate amounts of this tissue (˜0.5 mg, 1 mg, 2.5 mg, 5 mg, 7.5 mg, and 10 mg) after thawing may be accurately weighed. A lesion-free area of skin from the euthanized animals may be selected optically and the EPR spectra of this area may be determined with the surface loop resonator using the optimized parameter set obtained from optimizing in vivo EPR techniques to detect primary cutaneous melanotic lesions. A small flap may be raised and incision may be made at depths of either 2, 5, or 8 mm and accurately measured. The FMH samples may then be placed at the base of the incision and the flap temporarily closed with thin surgical tape. The EPR spectrum of the implanted tissue may then be obtained with the surface coil. The tape may be removed, the tissue removed, and the next largest tissue sample may be implanted. This overall experiment may be performed with 5 more replicates with analysis of the results of each. Should analysis indicate it, one may increase the number of replicates. The absolute signal intensity and the signal: noise ratio of each spectrum may be measured automatically by the Xepr software.

The signal intensity and signal: noise ratio (both ratio variables) may be analyzed as functions of FMH tissue mass and the depth to which it was implanted to determine the minimal detectable mass of FMH at differing depths. For example, a signal:noise ratio of greater than 2 may be used. This provides definitive quantitative measure of how useful this approach is to EPR virtual biopsy.

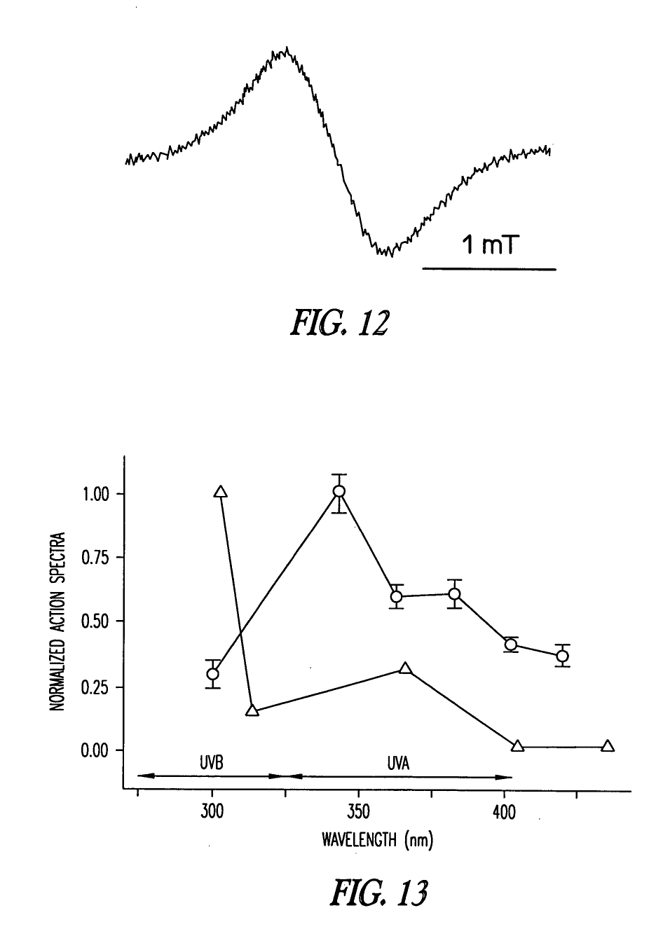

In an embodiment, EPR spectroscopy may be used in experiments on UVA induced melanin radicals. EPR may be applied to UVA induced melanin radicals in both mouse & fish skin to generate action spectra in models of melanoma causation. In an embodiment, experiments may provide EPR detection of UV-induced melanin radical species in ex vivo skin. Using Xe and Xe/Hg arc lamps with appropriate interference filters, action spectra for the generation of UV-induced melanin radical species in intact murine skin ex vivo may be generated utilizing EPR spectroscopy. FIG. 12 shows an EPR spectrum of UV-induced melanin radical in C57 murine skin using ex vivo UV illumination from a 300 W Xe arc lamp with a UVB/C blocking filter and a 280-400 nm dichroic mirror.

FIG. 13 shows a normalized action spectrum of UV-induced melanin radical (open circles) in C57 (pigmented) murine skin ex vivo at 37° C., (n=5±S.D.) together with the action spectrum of melanoma induction in Xiphophorus (open triangles). The action spectrum may be obtained by mounting 3 mm diameter punch biopsies in a custom designed EPR tissue holder in a Bruker Elexsys system and irradiating the biopsies with light from a 300 W Xe arc. The light may be filtered with narrow band width interference filters via a fiber optic. Light output for each filtered wavelength may be characterized using a calibrated Optronic model 742 scanning spectroradiometer.

For comparison, the action spectrum for UV induced melanin radical formation is shown in FIG. 13 together with the action spectrum for melanoma induction in Xiphophorus. It can be seen that there is substantial melanin radical generation in the mouse skin (that is also a measure of relative oxidant release from irradiated melanin,) that peaks in the UVA. This spectrum approximately provides a spectrum as would be expected by convolution of the action spectrum of in vitro melanin radical formation with the differential transmission of UV wavelengths to the epidermal/dermal junction. Similar data can be obtained for the ascorbyl radical, enabling a powerful insight into oxidative stress mechanisms and products in melanocytes in skin. The potential importance of correlating melanin and ascorbyl radical action spectra with an action spectrum of FMH and melanoma causation in M. domestica is significant. EPR spectroscopy is uniquely able to non-invasively determine and quantify these radical species in melanocytes in situ in intact skin. It is important to note that included are all of the most important parameters required for UVA.

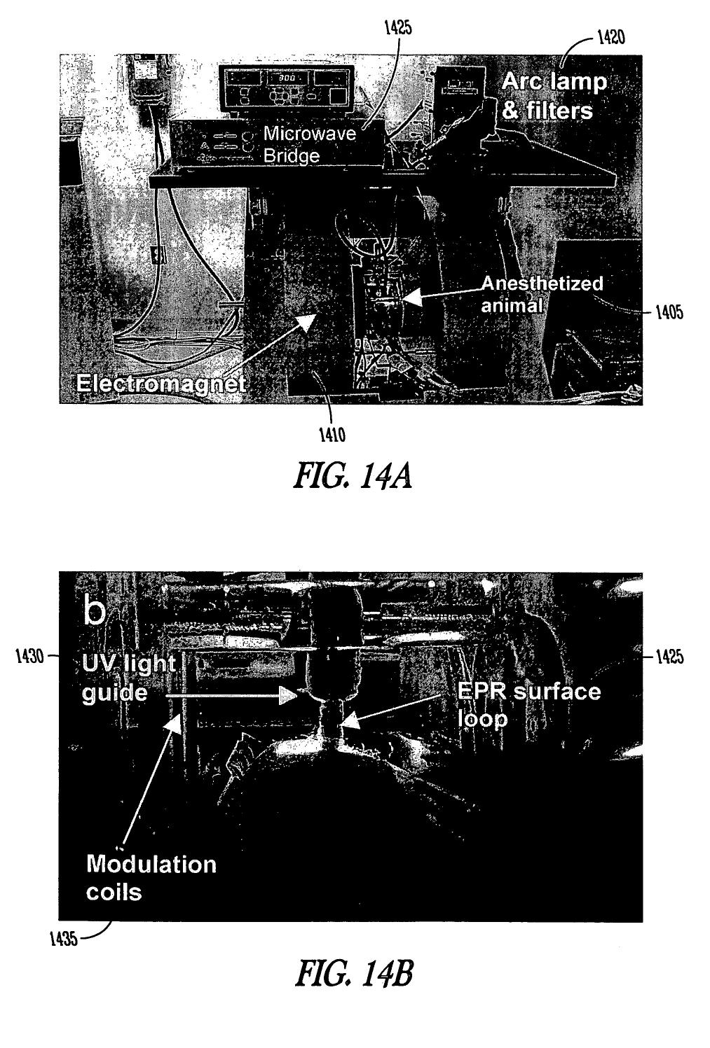

An embodiment includes in vivo, in situ detection of UV-induced radicals in skin. In vivo EPR technology has been developed by a number of groups over the last 15 years, with commercially available equipment available in the last 5 or so years. In an embodiment, through the use of a special microwave bridge operating at 1 GHz frequencies with an external loop EPR resonator, EPR detection may be performed in anesthetized, live animals, with UV irradiation via a fiber optic delivered to the skin inside the detection loop. FIGS. 14A and 14B illustrate In vivo EPR instrumentation for measurement of UV induced radicals. FIG. 14A depicts an overview of experimental setup showing positioning of an animal sample 1405 for irradiation. The experimental setup includes an electromagnet 1410, a microwave bridge 1415, and arc lamp & filters 1420. FIG. 14B depicts close up (from rear) showing anesthetized hairless mouse 1405 in position under a detection loop 1425 with UVA irradiation via a liquid light guide 1430. The experimental setup includes modulation coils 1435.

In various embodiments including various experiments, methods and apparatus use electron paramagnetic resonance spectroscopy to provide a signal from melanin to image a melanoma, to detect metastatic melanoma in a sentinel lymph node, to measure light penetration in melanocytes in skin, or combinations thereof. In various embodiments, apparatus may include instructions for computer control of the apparatus to perform embodiments of the functions described herein. Instructions for such automated control and analysis made reside in various forms of computer readable medium.

Although specific embodiments have been illustrated and described herein, it will be appreciated by those of ordinary skill in the art that any arrangement that is calculated to achieve the same purpose may be substituted for the specific embodiments shown. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description.

1. A method comprising:
using electron paramagnetic resonance spectroscopy to provide a signal from melanin to image a melanoma, to detect metastatic melanoma in a sentinel lymph node, to measure light penetration in melanocytes in skin, or to provide a combination thereof.

2. The method of claim 1, wherein using electron paramagnetic resonance spectroscopy includes using electron paramagnetic resonance spectroscopy in vivo.

3. The method of claim 1, wherein using electron paramagnetic resonance spectroscopy includes using in vivo electron paramagnetic resonance techniques to detect primary cutaneous melanotic lesions.

4. A method comprising:
imaging a melanoma using an electron paramagnetic resonance signal from melanin endogenous to the melanoma.

5. The method of claim 4, wherein the method includes applying in vivo electron paramagnetic resonance techniques to use molecular contrast of the melanin.

6. The method of claim 4, wherein the method includes using a correlation between gross optically apparent tumor morphology and an electron paramagnetic resonance image of the melanoma.

7. The method of claim 4, wherein the method includes determining a tumor thickness estimation of the melanoma.

8. The method of claim 4, wherein using an electron paramagnetic resonance signal includes using an in vivo electron paramagnetic resonance signal to detect primary cutaneous melanotic lesions.

9. The method of claim 8, wherein the method includes using 1 GHz spectroscopy with surface and whole body coils to detect the electron paramagnetic resonance signal in melanotic lesions.

10. The method of claim 4, wherein the method includes imaging a depth of focal melanocytic hyperplasia.

11. The method of claim 4, wherein the method includes applying magnetic field gradients to provide spatial imaging of melanoma depth and margins.

12. The method of claim 4, wherein the method includes performing an electron paramagnetic resonance virtual biopsy of tissue.

13. The method of claim 12, wherein performing an electron paramagnetic resonance virtual biopsy of tissue includes performing an electron paramagnetic resonance virtual biopsy of lymph nodes.

14. The method of claim 4, wherein the method includes detecting and imaging melanoma at depths ranging from 0.5 mm to 10 mm from skin surface.

15. The method of claim 4, wherein the method includes detecting and imaging melanoma at depths of 10 mm or greater from skin surface.

16. A method comprising:
detecting melanoma in a sentinel lymph node using electron paramagnetic resonance spectroscopy.

17. The method of claim 16, wherein detecting melanoma includes determining the status of the sentinel lymph node in less than about 20 minutes.

18. The method of claim 16, wherein detecting melanoma includes using properties of an electron paramagnetic resonance signal for melanin.

19. The method of claim 18, wherein using properties of a electron paramagnetic resonance signal for melanin include using one or more of a g value, linewidth and shape, power saturation properties.

20. The method of claim 16, wherein the method includes using electron paramagnetic resonance spectroscopy to image a depth of a pre-melanoma lesion focal melanocytic hyperplasia.

21. The method of claim 16, wherein the method includes using electron paramagnetic resonance spectroscopy to image a depth of a malignant melanoma.

22. The method of claim 16, wherein the method includes automatically assessing a signal provided by an electron paramagnetic resonance spectroscopy device using a database containing properties associated with a melanin signal.

23. The method of claim 16, wherein detecting melanoma includes using electron paramagnetic resonance spectroscopy on excised tissue.

24. The method of claim 16, wherein using electron paramagnetic resonance spectroscopy includes using electron paramagnetic resonance spectroscopy in situ on tissue.

25. A method comprising:
using electron paramagnetic resonance spectroscopy to measure a UV induced radical signal from melanin to measure light penetration in melanocytes in skin.

26. The method of claim 25, wherein the method includes determining effectiveness of a sunscreen to protect against melanoma based on the UV induced radical signal from melanin.

27. The method of claim 25, wherein the method includes assigning a melanoma prevention factor to a sunscreen.

28. The method of claim 25, wherein the method including formulating a sunscreen having a high melanoma prevention factor.

29. An apparatus comprising:
an electron paramagnetic resonance spectroscopy unit; and a
analyzing unit to analyze an electron paramagnetic resonance signal provided by the electron paramagnetic resonance spectroscopy unit to identify melanin to provide melanoma related information, the melanoma related information to image a melanoma, to detect metastatic melanoma in a sentinel lymph node, or to measure light penetration in melanocytes in skin.

30. The apparatus of claim 29, wherein the analyzing unit includes a database containing properties associated with a melanin signal to automatically assess the electron paramagnetic resonance signal.

31. The apparatus of claim 30, wherein the properties include one or more of a g-value, linewidth and shape, or power saturation properties.

32. The apparatus of claim 29, wherein the electron paramagnetic resonance spectroscopy unit includes a microwave bridge with an external loop electron paramagnetic resonance resonator.

33. The apparatus of claim 32, wherein the microwave bridge is configured to operate at 1 GHz.

34. The apparatus of claim 29, wherein the apparatus includes a UV irradiation source.

35. The apparatus of claim 34, wherein the UV irradiation source includes a fiber optic device.

36. The apparatus of claim 34, wherein the analysis unit is adapted to assign a melanoma prevention factor to a sunscreen.

37. The apparatus of claim 29, wherein the electron paramagnetic resonance spectroscopy unit and the analyzing unit are configured to provide an image of a melanoma.

38. The apparatus of claim 37, wherein the electron paramagnetic resonance spectroscopy unit and the analyzing unit are configured to provide an image of a melanoma having a depth of 0.5 mm to 0.10 mm from a skin surface.

39. The apparatus of claim 37, wherein the electron paramagnetic resonance spectroscopy unit and the analyzing unit are configured to provide an image of a melanoma having a depth of 0.10 mm or more from a skin surface.

40. The apparatus of claim 29, wherein the electron paramagnetic resonance spectroscopy unit and the analyzing unit are configured to detect metastatic melanoma including a determination of the status of a sentinel lymph node.

41. The apparatus of claim 40, wherein the determination of the status of a sentinel lymph node includes the determination of the status of a sentinel lymph node in less than about 20 minutes.

42. The apparatus of claim 29, wherein the apparatus includes a computer readable medium that stores instructions, which when performed by the apparatus, cause the apparatus to perform one or more of imaging a melanoma, detecting melanoma in a sentinel lymph node, or measuring light penetration in melanocytes in skin, using the electron paramagnetic resonance spectroscopy unit.

43. A computer readable medium that stores instructions, which when performed by a machine, cause the machine to use electron paramagnetic resonance spectroscopy to provide a signal from melanin to image a melanoma, to detect metastatic melanoma in a sentinel lymph node, to measure light penetration in melanocytes in skin, or to provide a combination thereof.

44. The computer readable medium of claim 43, wherein the computer readable medium stores instructions to control an electron paramagnetic resonance spectroscopy unit.

45. The computer readable medium of claim 43, wherein the computer readable medium stores instructions to automatically determine status of the sentinel lymph node.

46. The computer readable medium of claim 43, wherein the computer readable medium stores instructions to assign a melanoma protection factor to a sunscreen.

47. The computer readable medium of claim 43, wherein the computer readable medium stores instructions to perform an electron paramagnetic resonance virtual biopsy of a lymph node.
